(19) United States
(12) Patent Application Publication
(10) Pub. No.: US 2005/0113811 A1
(43) Pub. Date: May 26, 2005

(54) Method and devices for treating ischemic congestive heart failure

(76) Inventors: Russell A. Houser, Livermore, CA (US); Arthur A. Bertolero, Danville, CA (US); Lon Annest, Tacoma, WA (US); Bill Hare, Princeton, NJ (US); Tamer Ibrahim, Oakland, CA (US); Steve Geyster, Milton, MA (US); Wendel Smith, Tacoma, WA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/825,786

(22) Filed: Apr. 16, 2004

Related U.S. Application Data
(63) Continuation-in-part of application No. 10/785,486, filed on Feb. 24, 2004.
Application No. 10/785,486 is a continuation of application No. 10/224,659, filed on Aug. 21, 2002.
(63) Continuation-in-part of application No. 10/183,396, filed on Jun. 28, 2002, now Pat. No. 6,726,696.
Application No. 10/183,396 is a continuation-in-part of application No. 10/127,714, filed on Apr. 23, 2002, now abandoned.
(60) Provisional application No. 60/466,653, filed on Apr. 29, 2003.
(60) Provisional application No. 60/485,568, filed on Jul. 7, 2003.
(60) Provisional application No. 60/488,292, filed on Jul. 18, 2003.
(60) Provisional application No. 60/499,946, filed on Sep. 2, 2003.
(60) Provisional application No. 60/500,762, filed on Sep. 4, 2003.
(60) Provisional application No. 60/512,293, filed on Oct. 17, 2003.
(60) Provisional application No. 60/518,270, filed on Nov. 5, 2003.
(60) Provisional application No. 60/534,514, filed on Jan. 6, 2004.
(60) Provisional application No. 60/286,269, filed on Apr. 24, 2001.
(60) Provisional application No. 60/300,892, filed on Jun. 25, 2001.
(60) Provisional application No. 60/302,255, filed on Jun. 28, 2001.

(51) Int. Cl. A61B 17/00

(52) U.S. Cl. 606/1; 606/151; 623/11.11; 128/898

(57) ABSTRACT

A kit and method are described for treating congestive heart failure. The kit may comprise multiple components including a shaping device, deployment tool, patch, and suture. The method may utilize one or more of these components.

### RELATED APPLICATIONS

This application claims priority from the following U.S. Provisional Patent Applications each of which is incorporated herein in its entirety by reference: Ser. No. 60/466,653, filed Apr. 29, 2003 and titled Ventricular Restoration; Ser. No. 60/485,568, filed Jul. 7, 2003 and titled Systems, Devices and Methods of Use for Treating Congestive Heart Failure (CHF); Ser. No. 60/488,292, filed Jul. 18, 2003 and titled Ventricular Sizing & Shaping Device and Method; Ser. No. 60/499,946, filed Sep. 2, 2003 and titled System and Method of Use to Employ Imaging Technology for Diagnosis, Measurement, Standardization, and Follow-up of Disease Processes and Determine Optimal Treatment; Ser. No. 60/500,762, filed Sep. 4, 2003 and titled Shaping Suture Device and Method of Use; Ser. No. 60/512,293, filed Oct. 17, 2003 and titled Less Invasive CHF Treatment—Reshaping the Heart; Ser. No. 60/518,270, filed Nov. 5, 2003 and titled Methods and Devices for Tracking Acute Myocardial Infarction; and Ser. No. 60/534,514, filed Jan. 5, 2004 and titled Squeeze Patch. This application also claims priority from and is a continuation-in-part from co-pending U.S. patent application Ser. No. 10/785,486, filed Feb. 17, 2004 and titled Patches and Collars for Medical Applications and Methods of Use, which claims priority from and is a continuation from U.S. patent application Ser. No. 10/224,659, filed Apr. 23, 2002 and titled Arteriotomy Closure Device and Techniques, which claims priority from U.S. Provisional Patent Application Ser. No. 60/286,269, filed Apr. 24, 2001 and titled Percutaneous Vessel Access Closure Device and Method; from U.S. Provisional Patent Application Ser. No. 60/300,892, filed Jun. 25, 2001 and titled Percutaneous Vessel Access Closure Device and Method; and from U.S. Provisional Patent Application Ser. No. 60/302,255, filed Jun. 28, 2001 and titled Percutaneous Vessel Access Closure Device and Method (Hemostatic Patch or Collar) each of which is incorporated herein in its entirety by reference. This application also claims priority from and is a continuation-in-part from co-pending U.S. patent application Ser. No. 10/183,396, filed Jun. 28, 2002 and titled Patches and Collars for Medical Applications and Methods of Use, which claims priority from and is a continuation-in-part from U.S. patent application Ser. No. 10/127,714, filed on Apr. 23, 2002, which claims priority from U.S. Provisional Patent Application No. 60/286,269, filed Apr. 24, 2001 and titled Percutaneous Vessel Access Closure Device and Method; from U.S. Provisional Patent Application Ser. No. 60/300,892, filed Jun. 25, 2001 and titled Percutaneous Vessel Access Closure Device and Method; and from U.S. Provisional Patent Application Ser. No. 60/302,255, filed Jun. 28, 2001 and titled Percutaneous Vessel Access Closure Device and Method (Hemostatic Patch or Collar), each of which is incorporated herein in its entirety by reference.

### BACKGROUND OF THE INVENTION

1. Field of the Invention

This application relates generally to medical devices and methods and more specifically to devices and methods for treating congestive heart failure.

2. Description of the Related Art

Congestive heart failure affects 5 million people in the United States, and the NIH reports that 550,000 new cases are diagnosed every year (U.S.). World-wide, the figure is estimated at 22 million. Death rates have grown at an almost exponential rate. Congestive heart failure is the most common discharge diagnosis among Americans over age 65.

Congestive heart failure is a clinical syndrome with heterogeneous etiologies including ischemic cardiomyopathy, valve dysfunction, hypertensive cardiomyopathy, chemotherapy, alcohol abuse, radiation injury, idiopathic conditions, and others. Therapy is directed at the underlying cause, such as coronary revascularization, valve replacement, bi-ventricular pacing, and extensive drug usage, leveled at both the source and the symptoms. Unfortunately, the collective results of all available therapies in the treatment of congestive heart failure are disappointing. Pharmacology and electrical resynchronization have improved the symptoms in many cases, but direct approaches to improving the function of the weakened heart muscle, the common thread in all cases, are few.

Congestive heart failure is a syndrome characterized by inadequate cardiac output, regardless of primary cause. One common cause of congestive heart failure is a previous heart attack causing “ischemia,” or lack of oxygen to the heart tissue. Responsible for approximately two-thirds of congestive heart failure patients, ischemic cardiomyopathy follows a predictable course. Initially, there is an index event, most commonly an anterior myocardial infarction. When treated, the patient is stabilized, often receiving a balloon catheter dilitation, intra-coronary stent or bypass graft, and has an initially unremarkable recovery. However, over the next one to three years, a process known as “ventricular remodeling” takes place where the previously conical chamber becomes spherical and substantially dilated, and previously normal segments become acontractile. The syndrome of disabling, chronic, congestive heart failure begins. Drugs such as ARBs (angiotensin receptor blockers) and ACE (angiotensin converting enzyme) inhibitors have been shown to retard the progress of this disintegration of cardiac function, but the end result is delay, not cure.

One common symptom of many classes of heart disease is enlargement of the heart and/or dilation of the left ventricle. The cause of ventricular dilation is typically the result of a chronic volume overload or specific damage to the myocardium. If portions of the myocardium are damaged, increased requirements are put on the remaining healthy myocardium such that the heart may attempt to compensate with ventricular dilation and muscle hypertrophy. In diseased hearts, the compensation is not sufficient and the ventricular dilation and muscle hypertrophy progress to a point where efficiency of heart function begins to fall. Further attempts by the heart to compensate may accelerate this reduction in efficiency.

One surgical approach, the Dor Procedure (endoventricular circular patch plasty), has improved the course of the disease in selected congestive heart failure victims by excluding and reinforcing the dysfunctional, or akinetic, portion of the ventricle. That procedure typically involves the following steps:
Define the infarcted area on ventricular wall;
Incise through the infarcted area into the ventricle;
Open and secure the flaps of scarred ventricular tissue that were created during the incision;
Define the border around the viable and infarcted tissue in the ventricular wall; and
Place a Fontan stitch or purse-string suture around the circumferential margin where viable tissue meets the infarcted tissue and tighten the stitch like a noose, drawing the viable tissue closer together. (A second row of sutures may be required for further size reduction.)

Optionally, the Dor Procedure may also involve suturing a patch of material (typically woven or knitted Dacron®, but others can also be used) on the inside of the ventricle, eliminating the defect in the ventricular wall defined by the tightened purse-string or strings.

While the Dor Procedure has benefits, it also has a few disadvantages. First, it is difficult for surgeons using the procedure to reshape the ventricle to its naturally elongated shape. The procedure tends to result instead in a spherically shaped ventricle. Without the elongated, conical shape normally associated with a healthy heart, the ventricle cannot perform the twisting motion at the apex that can account for a large percentage of the pumping action. A more spherical ventricle must rely almost entirely on lateral squeezing action, which is very inefficient.

In addition, the Dor Procedure requires surgeons to estimate the appropriate ventricle size and shape for a particular patient. Some surgeons inaccurately estimate the appropriate ventricle size resulting in a ventricle that is too small, which may leave the patient clinically worse than before the procedure. Other surgeons fail to account for the desired shape of the ventricle and do nothing to try to achieve a less spherical shape.

For the past several years, Dr. Dor has attempted to decrease the likelihood of achieving the result of an inappropriately small ventricle through using a fluid filled balloon as a guide for the practitioner when drawing the tissue together. The use of a balloon, however, has not adequately solved the problem. First, it does not aid the practitioner in achieving a less spherically shaped ventricle. Second, the practitioner must still estimate the appropriate size for the ventricle in deciding how much to fill and expand the balloon. Finally, the balloon has the added disadvantage that a needle or any other sharp object used during the procedure may rupture the balloon and render it useless for the remainder of the procedure.

### SUMMARY OF THE INVENTION

The present invention endeavors to address those deficiencies as well as improve and enhance the overall treatment of an ischemic heart. In one embodiment, the present invention comprises a kit comprising multiple components, as well as a method for providing and/or utilizing one or more of the components, for treating ischemic congestive heart failure. This kit and the method of providing and using the kit can aid a practitioner in excluding and reinforcing the akinetic portion of a heart chamber, a procedure sometimes referred to as Surgical Ventricular Restoration (SVR), without creating a heart chamber that is too small or too spherical.

In one embodiment of the invention, the kit comprises a device for sizing and shaping a deficient ventricle. One benefit of certain embodiments of the shaping device of this invention, discussed in more detail below, is that they do not require inflation. Unlike inflatable shaping devices, there is no risk of puncturing and deflating this device during the procedure. The shaping device of this invention can also be compliant unlike inflatable devices that must be inflated to a point at which they become non-compliant. The kit may also comprise a device for deploying and removing the shaping device.

The kit further may comprise a patch having one or more inventive features that may be used with or without the shaping device to help secure the opening in the ventricle used to exclude akinetic tissue. The kit may also comprise a device for deploying the patch. The kit may further comprise a shaping suture used to more effectively exclude akinetic tissue and close the incision in the ventricle.

The method of the present invention may comprise steps that utilize one or more of the following components: a shaper, a patch, and a shaping suture. These components can aid in creating a heart chamber of the appropriate shape and size. The present method may comprise the step of determining an appropriate size and shape for a heart chamber based on the needs of the patient. A practitioner may use any combination of methods for determining the appropriate heart chamber size and shape for the patient. Some potential methods include but are not limited to magnetic resonance imaging (MRI), PET Scan, Echo, ultrasound, end diastolic volume, and/or body surface measurement. Images of the heart chamber may be provided to a computer. Computer software, databases, or computer networks may aid in determining an appropriate size and shape as well. The practitioner may then choose a correctly sized shaping device, suture, and/or patch for the patient.

In one application, the present method comprises the steps of identifying the infarct area of a heart chamber wall and making an incision through the infarct tissue. The infarct tissue comprises at least one of the following: akinetic or dyskinetic tissue that is dead, unhealthy, or otherwise sub-optimal. The practitioner may identify the infarct area through any number of methods including but not limited to the following: drawing a vacuum in the ventricle thereby causing the infarcted area to be revealed as an area that is depressed relative to the surrounding healthy tissue; palpation; or any other appropriate method. The step of making an incision may be performed in an open-heart procedure or a more minimally invasive procedure such as with an endoscope with an incising tip.

The method further comprises the step of inserting a shaping device through the incision and into the heart chamber. As discussed in detail below, the shaping device may be compressed for insertion and then allowed to expand once inside the heart chamber.

The method may further comprise the step of weaving a purse-string stitch around the border between the akinetic or dyskinetic tissue and the healthier tissue. In weaving the purse-string stitch, the practitioner preferably excludes the akinetic tissue and reshapes the heart chamber by pulling the chamber wall together around the shaping device. The practitioner may use more than one row of purse-string stitches as needed. Preferably, the practitioner can use a shaping suture to weave the purse-string stitch, which can aid in forming an oblong rather than circular shape when pulling tissue together with the purse-string stitch.

The method may further comprise the steps of removing the shaping device, inserting a patch, and securing it to the inner wall of the heart chamber. The step of securing the patch to the heart chamber may include, but is not limited, to applying adhesive, weaving a purse-string or other type of suture through the patch, or engaging barbs or other protrusions, etc. from the patch. The patch is preferably sized and shaped for the area to which it is applied. The patch also may comprise a rim comprising a shape memory material to aid in forming and possibly maintaining an appropriately sized and shaped heart chamber. The method may further comprise the step of stitching the myocardium and epicardium closed over the patch.

For purposes of summarizing the invention, certain aspects, advantages and novel features of the invention have been described herein. Of course, it is to be understood that not necessarily all such aspects, advantages or features will be embodied in any particular embodiment of the invention.

### BRIEF DESCRIPTION OF THE DRAWINGS

FIG. 1A is a cross sectional view illustrating a weakened heart chamber before reconstruction. FIG. 1B is a cross sectional view of a heart chamber illustrating one embodiment of the kit of this invention as employed in a procedure to reconstruct a heart chamber. FIG. 1 C is a cross sectional view illustrating a heart chamber after reconstruction.

FIG. 2 is a perspective view of one embodiment of the shaping device of this invention.

FIG. 3 is a perspective view of one embodiment of the shaping device comprising removable sections.

FIG. 4 is a perspective view of one embodiment of the shaping device of this invention designed asymmetrically to fit an anatomical structure.

FIG. 5 is a perspective view of one embodiment of the shaping device of this invention comprising reinforcing wires.

FIG. 6 is a perspective view of one embodiment of the shaping device of this invention comprising one or more holes to affect flexibility or other physical properties.

FIG. 7 is a side view illustrating one embodiment of the shaping device of this invention along with an embodiment of a deployment device. FIG. 7A is a cutaway view of the deployment device illustrated in FIG. 7, showing an inner and outer sheath.

FIG. 8 is a side view illustrating one embodiment of the shaping device of this invention along with an embodiment of a deployment device and an external organ vacuum.

FIG. 9 is a front view illustrating one embodiment of the patch of this invention.

FIG. 10 is a front view illustrating another embodiment of the patch of this invention.

FIG. 11A is a cross sectional view of one embodiment of the patch of this invention illustrating a patch comprising a single layer. FIG. 11B is a cross sectional view of one embodiment of the patch of this invention illustrating a patch comprising two layers. FIG. 11C is a cross sectional view of one embodiment of the patch of this invention illustrating a patch comprising three layers.

FIG. 12A is a perspective view of one embodiment of the patch of this invention illustrating a patch applied to the outside of a heart chamber. FIG. 12B is a front view of one embodiment of the patch of this invention illustrating a patch comprising a plurality of arms and a base. FIG. 12C is a perspective view of one embodiment of the patch of this invention illustrating a patch comprising a plurality of arms and a base.

FIG. 13 illustrates one embodiment of the shaping suture of this invention.

FIG. 14 is a fragmentary view illustrating in greater detail portions of one embodiment the shaping suture illustrated in FIG. 13.

FIG. 15 illustrates one the beginning of a purse-string stitch using one embodiment of the shaping suture of this invention.

FIG. 16 illustrates using a crimping tool to attach exposed segments of nitinol when using one embodiment of the shaping suture of this invention.

FIG. 17 is a side view of one embodiment of the integrated sizer/shaper and patch of this invention.

FIG. 18 is a side view of one embodiment of the patch sizing template of this invention. FIG. 18A is a front elevation view of the template member of the patch sizing template device shown in FIG. 18, removed from the handle.

FIG. 19 is a side view of another embodiment of the patch sizing template of this invention.

### DETAILED DESCRIPTION OF THE PREFERRED EMBODIMENT

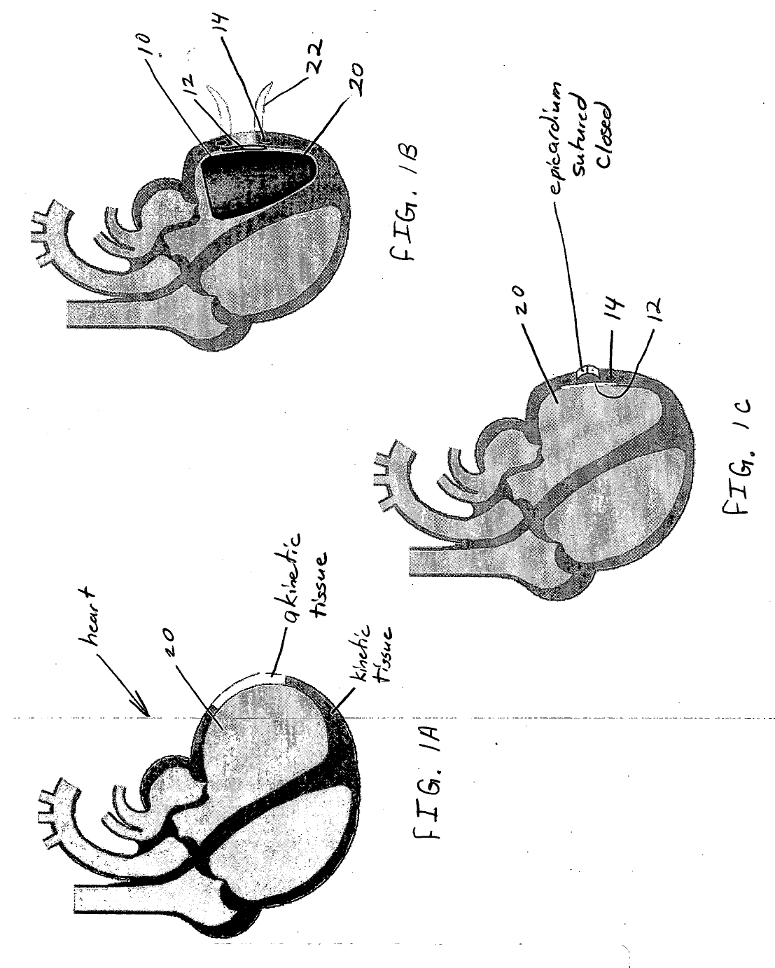

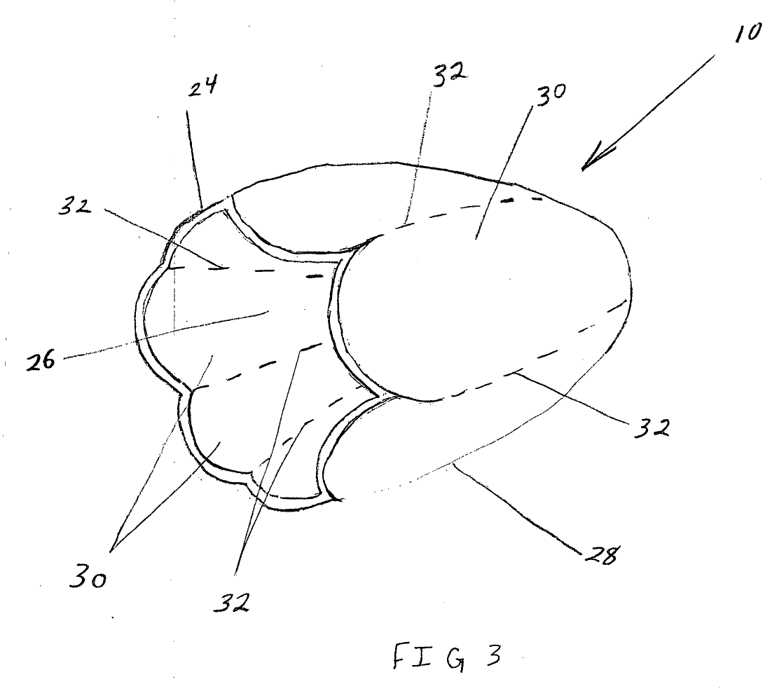

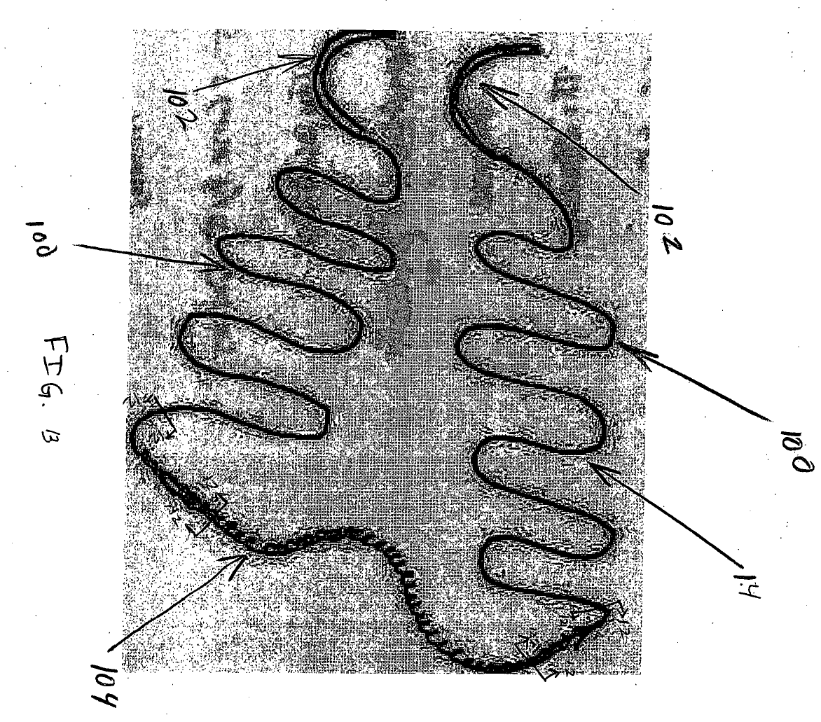

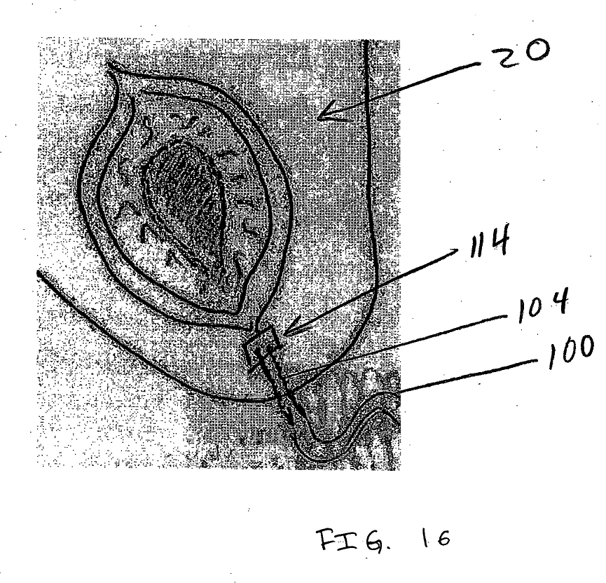

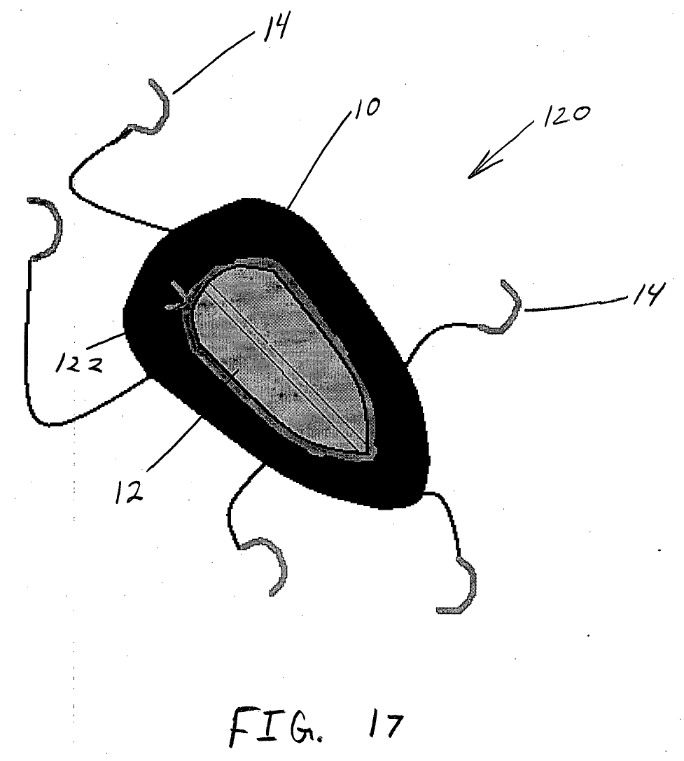

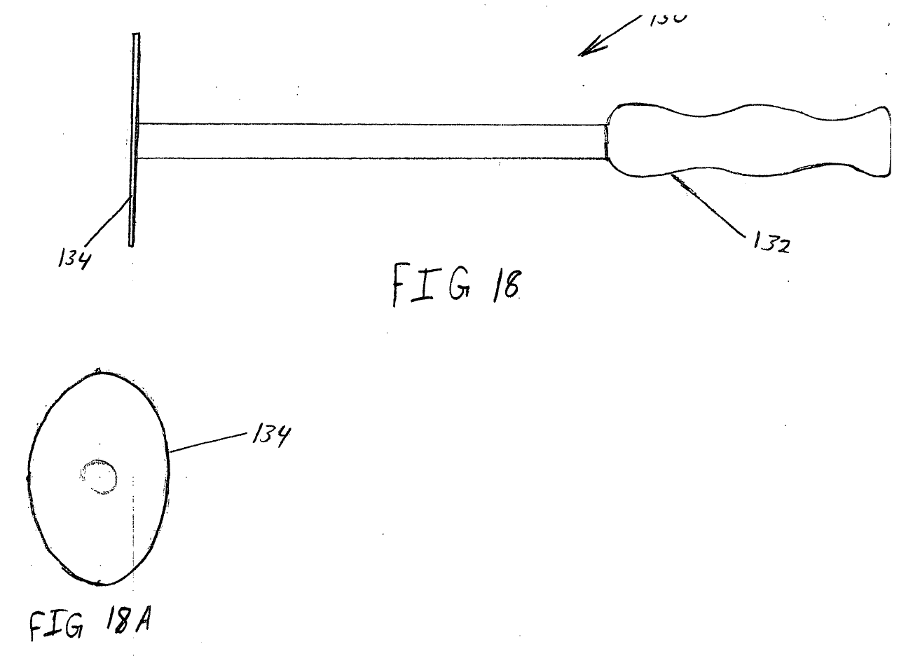

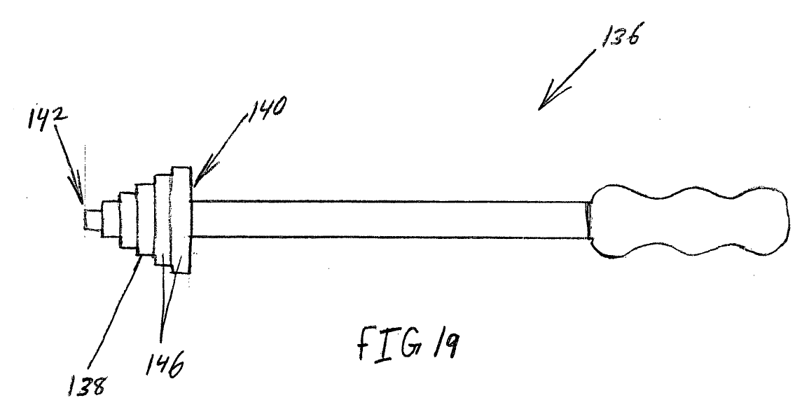

The present invention allows more exact execution of a procedure or process whose purpose is to create a more optimum size and shape of an organ, such as the heart or more particularly a heart chamber, or another structure undergoing reconstruction. For example, one such procedure is known as Surgical Ventricular Restoration (SVR). Referring to FIGS. 1A-1C, the invention comprises a kit and a method of using the kit. The kit may include a shaping device 10, described in more detail below in association with FIGS. 3-7. In addition, the kit may include a patch 12, described in more detail below in association with FIGS. 8-11. The kit may further include a shaping suture 14, described in more detail below in association with FIGS. 12-15. In some embodiments, the kit may also include an integrated shaping device and patch, described in more detail below in association with FIG. 16. Some embodiments of the kit may further include a patch sizing template, described below in more detail in association with FIGS. 17-18.

Referring again to FIGS. 1A-1C, the reconstruction of a heart chamber using the kit and the method of this invention may be described. Referring to FIG. 1A, before reconstruction, the heart chamber, in this case the left ventricle 20, is abnormally dilated and has acquired a spherical shape. A portion of the ventricle wall has become nonfunctioning or akinetic or dyskinetic. As used throughout this application, the term akinetic means dyskinetic, injured, weakened, nonfunctioning, dead, akinetic, or otherwise damaged. According to one application of the method of this invention, an incision is made within the akinetic tissue. Referring to FIG. 1B, the shaping device 10 is then inserted in the ventricle. Using the shaping suture 14, the practitioner weaves a purse-string stitch to exclude the akinetic tissue and to bring the ventricle wall against the shaping device 10. The patch 12 may also be used to aid in shaping the ventricle or to close or to aid in closing the incision. The shaping device 10 is removed, and the incision is closed. FIG. 1C illustrates the resulting, reconstructed left ventricle.

In an alternate application of this method, the practitioner may insert a catheter or other deployment device into the patient's atrium. The practitioner then guides the device through the mitral valve into the patient's left ventricle. The shaping device 10 and/or patch 12 may be inserted through the catheter (or other device) or be part of the device such as a balloon. A shaping suture 14, clamping patch 12, or other device may be used to bring the wall of the left ventricle against the shaping device 10. The shaping suture 14, patch 12, or other device may be on the inside or the outside of the heart chamber wall, or both, and may comprise a temporary and/or a permanent device. In addition, the shaping device 10 may be used to bring the wall of the left ventricle against the patch 12 thereby deploying barbs, protrusions, or another device designed to aid in holding the patch 12 in place.

In one embodiment of the kit of this invention, one or more of the devices in the kit may be sized for a particular patient according to the size of another device in the kit. For example, the kit may comprise a patch 12 of a particular size according to the size of the shaping device 10 included in the kit. Alternatively, the kit may comprise a shaping device 10 of a particular size according to the patch 12 included in the kit. The shaping suture 14, if included, may also be sized according to the patch 12 and/or the shaping device 10. These are merely a few examples, in other embodiments different sizing relationships may be used.

Sizing/Shaping Device

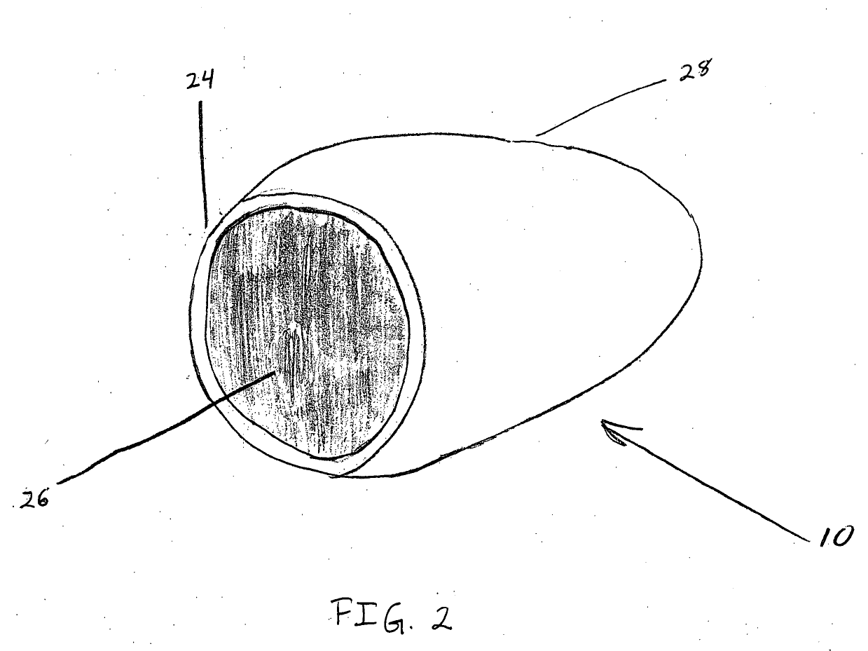

Referring to FIGS. 2-7, the shaping device 10 can be placed. inside an organ to guide a practitioner performing a procedure to repair or reconstruct the organ. In one application, the shaping device 10 may be used as a guide or template to guide surgical or alternative reconstruction to what has been predetermined to be a more optimum size and/or shape of the left ventricle 20 and/or for surgically reducing the size of the ventricle 20. In this application, the shaping device 10 may act as an idealized anatomical shaper and helps the surgeon to know how much of the ventricle 20 to bring together for a more optimum size and shape, without the risk of making the resulting ventricle too small.

The shaping device 10 can help to improve the resulting size and shape of the organ according to the requirements of a particular patient. A detailed pre-reconstruction analysis based on characteristics of the dysfunctional structure and those of the normal/optimal state may be conducted to aid in choosing the appropriate size and shape for the shaping device 10. Thus, the use of the shaping device 10 as a guide can help to eliminate the mistakes that occur when a practitioner relies only on his judgment to estimate the appropriate size and shape.

The shaping device 10 may be utilized during an open field or minimally invasive surgical procedure. It may also be deployed through a standard or modified endoscope. The shaping device 10 may also be used for laparoscopic, robotically assisted and/or percutaneous procedures. The shaping device 10 may be compressible and re-expandable to allow compression during insertion and withdrawal, and re-expansion once inserted into the organ. The ability to compress the shaping device 10 into a reduced cross section profile facilitates insertion and removal.

The shaping device 10 may have a stock size and shape or it may be made custom for a particular patient's anatomy. For example, the device may be available in multiple stock sizes (90, 110 and 130 cc or small, medium, and large, for example). If it is custom made for a particular patient, MRI, PET scan, Echo, ultrasound, any other visualization techniques, or any other appropriate method may be used to determine the pre-condition and/or optimum post-procedure size and shape of the ventricle, as described in more detail in the following provisional patent applications incorporated herein in their entirety by reference: U.S. Provisional Patent Application Ser. No. 60/466,653, filed on Apr. 29, 2003 and titled Ventricular Restoration; U.S. Provisional Patent Application Ser. No. 60/499,946, filed on Sep. 2, 2003 and titled System and Method of Use to Employ Imaging Technology for Diagnosis, Measurement, Standardization, and Follow-up of Disease Processes and Determine Optimal Treatment; and U.S. Provisional Patent Application Ser. No. 60/518,270, filed Nov. 5, 2003 and titled Methods and Devices for Tracking Acute Myocardial Infarction.

Referring to FIG. 2, the shaping device 10 may be tulip shaped or egg shaped. In alternate embodiments, the shaping device 10 may comprise a cone shape or any other suitable shape. The shaping device 10 may be symmetric or asymmetric, anywhere on the device, top, bottom and/or body. The top edge 24 of the shaping device 10 may be straight and flat, sinusoidal, a combination of these shapes, or made into another suitable shape or geometry.

In one embodiment, the shaping device 10 may have one or more reference marks. The reference marks may comprise a single mark, multiple marks, a grid, or any other appropriate markings. The reference marks may be used for orienting or positioning the shaping device 10 within the hollow body structure, for guiding the suture line, for determining whether a patch 12 is needed, for determining an appropriate size for the patch 12, for guiding the positioning of tissue, and/or for any other suitable purpose. These reference marks may be molded onto the shaping device 10 as indentations or raised areas. Alternately, the reference marks may be printed on or otherwise applied to the shaping device 10.

As shown in FIG. 2, in one embodiment the shaping device 10 may be hollow such that it partially or entirely encloses an interior space 26. In this embodiment, one or both ends of the shaping device 10 may be either covered, partially covered, or open. That can aid in preventing inadvertent expansion of the shaping device 10. In another embodiment, the shaping device 10 may have one or more cutouts and/or indentations so as not to damage structures such as papillary muscles, chordae tendinae, valves or valve structures including the annulus.

The outside 28 of the shaping device 10 may be smooth, textured, or a combination of both smooth and textured. In one embodiment, a lubricant, such as parylene, may be applied to the exterior and or interior of the shaping device 10. Additionally, the shaping device 10 may comprise holes, slots, or thin or weakened wall areas to initiate or focus the bending or folding during insertion and removal and to assist with insertion and removal. In another embodiment, the shaping device 10 may comprise “pods” on the surface connected to an airtight lumen or lumens that, when connected to suction, can enhance fixation or stabilization.

Referring to FIG. 3, in another embodiment the shaping device 10 may have one or more removable sections 30. A perforated or weakened area 32 can facilitate and/or guide the removal of one or more sections 30. By removing the one or more sections 30, a practitioner can adjust the size of the shaping device 10.

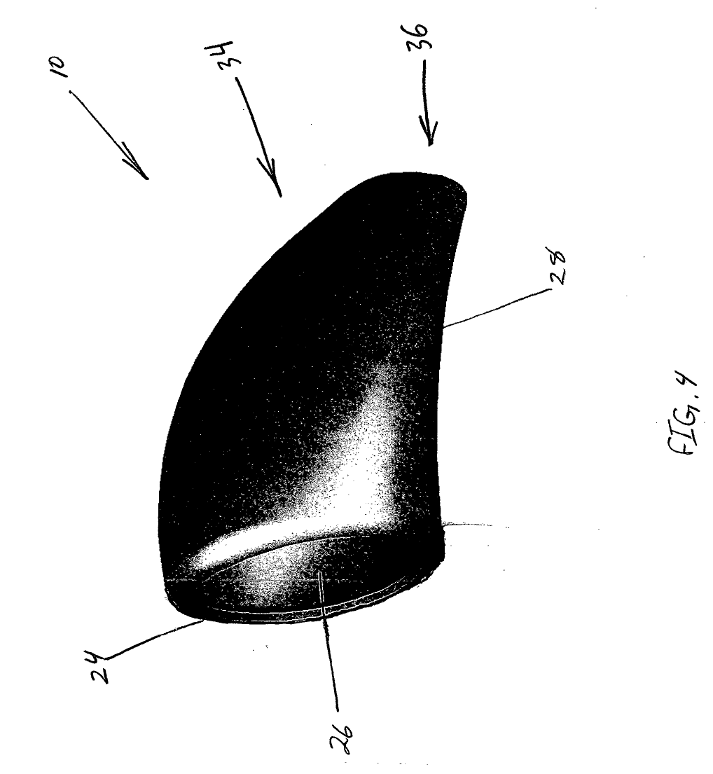

Referring to FIG. 4, the shaping device 10 may comprise one or more sections 34 designed to fit an anatomical structure, for example the area near the aortic outflow tract or mitral valve. In the embodiment shown in FIG. 4, the shaping device 10 is asymmetrical toward its distal end 36. The asymmetrical configuration may allow the shaping device 10 to better accommodate surrounding anatomical structures when in the correct position and orientation. It may also act as a guide to aid the practitioner in positioning and orienting the shaping device 10 within the heart chamber.

One embodiment of the compressible shaping device 10 may be covered with an airtight material and connected to a lumen for loading and deployment. A Luer, stopcock or another type of connector can be placed on the opposite end of the lumen from the device so that when a vacuum is created (by using a syringe or the vacuum supplied in the surgical suite, or any other appropriate source), the sponge or foam will collapse down to a reduced cross-sectional profile for insertion and removal from the ventricle (or other location). Once the vacuum has been removed, the sponge or foam shaping device 10 self expands to its natural size.

Because the shaping device 10 need not include a bladder or balloon component on the surface, it is less likely to be functionally impaired by suture, blade, or any other sharp instrument. However, in an alternate embodiment, the shaping device 10 may have an internal bladder that can be aspirated. A Luer-lock fitting with a syringe can be used for aspirating the bladder to control its shape and/or size.

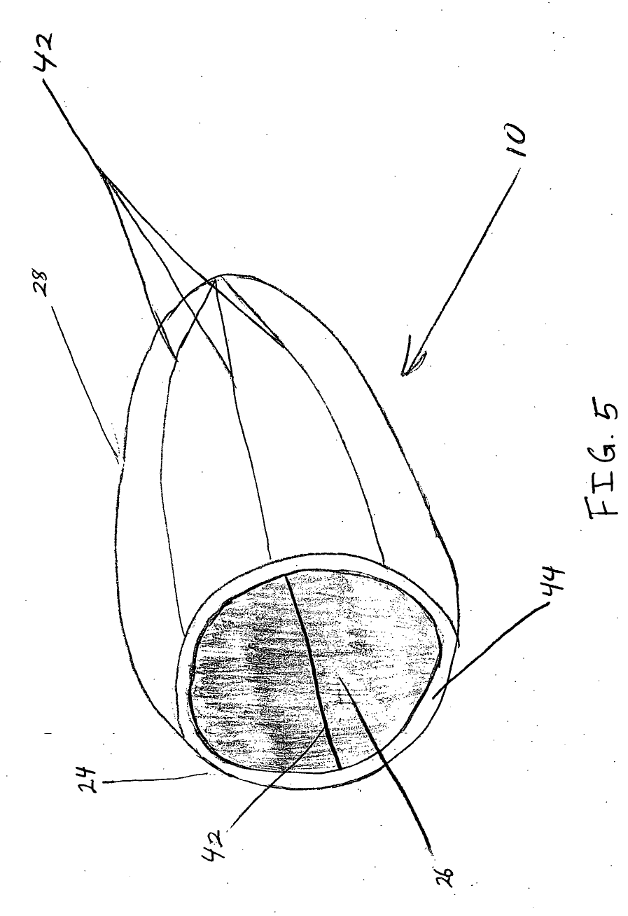

As shown in FIG. 5, the shaping device 10 may also include one or more reinforcing elements 42 to provide additional support. The reinforcing elements 42 may include one or more strips, sheets, wires, rods, tubes, mandrels, any combination of these. The reinforcing member or members 42 may be located on the inside surface of the walls 44 of the shaping device 10, on the outside of the walls 44, within the walls 44, or any combination of these locations. The inclusion of these components may assist with self-expansion of the device.

One method of constructing the shaping device 10 is through molding. Alternative construction methods may include stereo lithography, casting, sintering, weaving, extrusion, a dip coating process, spraying, laminating, a combination of any of these, or another suitable method or process.

In one embodiment, the shaping device 10 may comprise a superelastic or shape memory material, a material that is inherently resistant to permanent deformation or is processed to be resistant to permanent deformation. One such shape memory material is the superelastic metal alloy nitinol, but many other materials may be used including other superelastic metal alloys, or superelastic shape-memory polymers. The shape memory material may permit fabrication of a shaping device 10 that is collapsible to a smaller size for insertion and self-expands once released in the organ. Once the compressed device is no longer constrained, it can snap back into its fully expanded shape. The expansion of the shaping device 10 may be achieved by the inherent spring of the material as in a superelastic material. In other embodiments the expansion of the shaping device 10 may be achieved by raising the ambient or component temperature (direct heating or the body's heat) for a shape memory effect.

The shaping device 10 may comprise heterogeneous materials. For example, some portions may be softer and more compressible while others may be stiffer and smoother. That can help to reduce trauma during placement and removal.

In one embodiment, the shaping device 10 comprises polyurethane or polyethylene, but any suitable material may be used. In other embodiments the shaping device 10 may comprise any of the following: a metal, a metal alloy, a polymer, rubber, foam, a sponge, silicone, (including silicone polyether and silicone polycarbonate, etc.), ePTFE, Dacron®, a combination of these materials, or any of these materials combined with any other suitable material. The device may be partially or totally radio-opaque by adding material such as barium sulfate or bismuth tri-oxide or another suitable material. In other embodiments, any portion of the shaping device 10 may be partially or completely coated with a biocompatible material, such as parylene, expanded polytetrafluoroethylene (ePTFE), polyester, polyurethane, silicone, Dacron®, urethane, and/or a composite or combination of these or of another suitable material or materials.

The shaping device 10 may comprise a material that is either substantially translucent, substantially opaque, or a combination of both at various locations. In one embodiment, the shaping device 10 may comprise a material having a color that contrasts with the natural color of cardiac tissue. The contrasting color can help a practitioner to more easily visually distinguish between the shaping device 10 and the cardiac tissue.

In alternate embodiments, the shaping device 10 may include the use of a vacuum, protrusions, knurling, surface dimples or spheres, compliant coatings, raised bands and/or lines, horizontal rings or other designs and methods to assist with temporarily holding the device against tissue to prevent slippage while in use. The vacuum utility may be accomplished using lumens or tubes with ports that allow the suction to contact tissue. The lumens or tubes may be connected to a vacuum source at the proximal end of the device (perhaps on or near a handle), using at least one Luer or similar type of connector. The vacuum lumens or tubes may be independent or connected to a single proximal connector.

The shaping device 10 may also comprise a “leash” or “tether” element used to assist in retrieval from the ventricular cavity. The leash element may be made from a single- or multi-element string. The string may or may not be braided. Alternatively, the leash element may be made from any other suitable component and material. The leash element may be attached to the shaping device 10 during fabrication, or as a second process. This element may be attached internally such that when tension is applied, the remote site of attachment may invaginate and deform the shaping device 10 in a way that is advantageous for placement, removal, or other function. The leash may be connected at one or more locations, anywhere on or in the shaping device 10. The leash may be a stiff or partially flexible structure, or a combination of both.

In another embodiment, the shaping device 10 may comprise one or more holes that permit a practitioner to attach suture material or another suitable material to the shaping device to form a leash. Other embodiments may include more than one leash or handle to manipulate, stabilize, remove or otherwise employ the shaping device 10 in its intended function. The end of the leash may include a pull tab located on the leash end opposite from the shaping device 10.

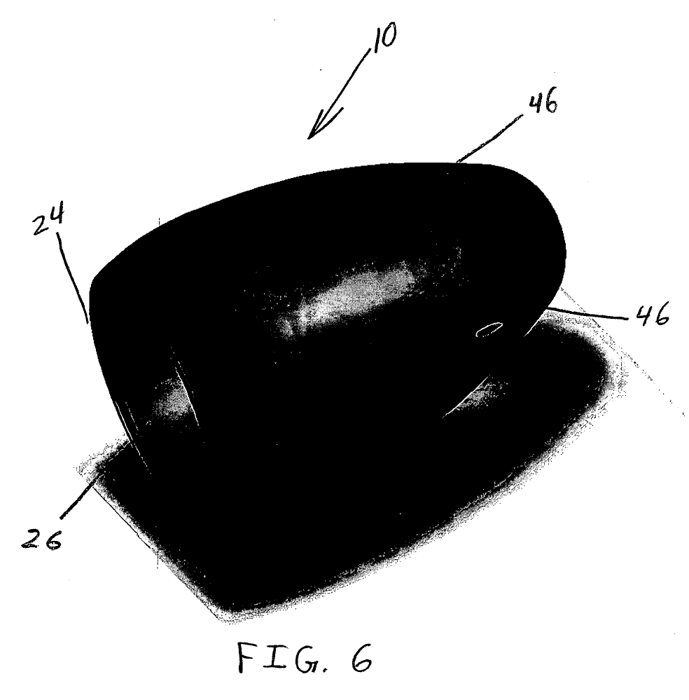

Referring to FIG. 6, in one embodiment the shaping device 10 may comprise one or more holes 46 to affect flexibility or other physical properties. In other embodiments, the shaping device 10 may comprise one or more slots or other piercings instead of, or in addition to, the one or more holes. Additionally at least one hole 46 may be used for and may enable venting, suction, and drainage during the procedure, something not possible when utilizing a balloon type sizing or shaping device. This process, known as the Smith-Luver Technique, is an important advantage in minimally invasive surgical ventricular restoration procedures.

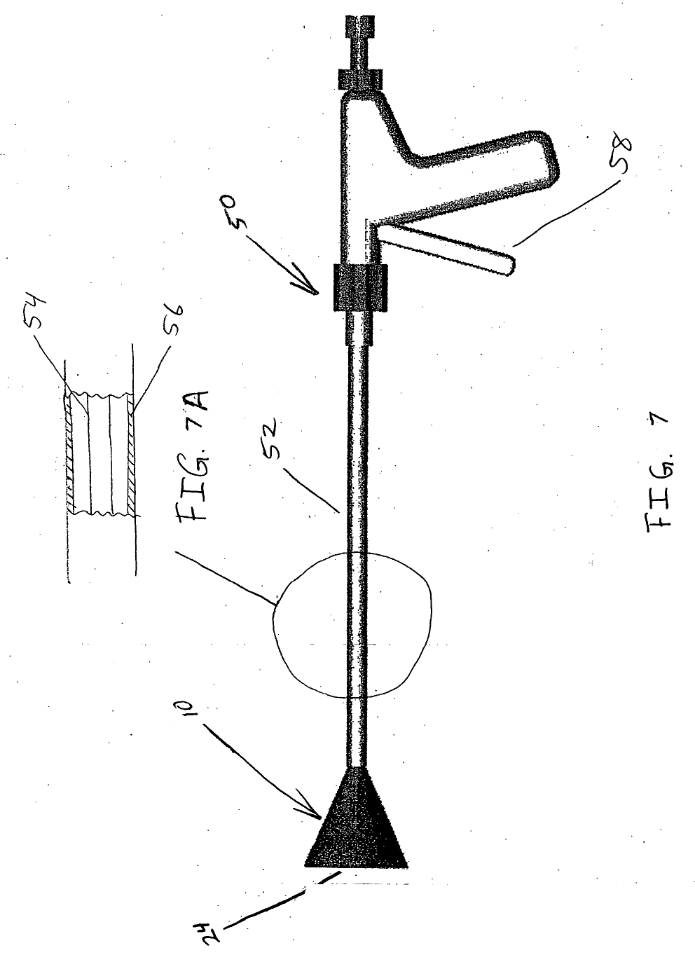

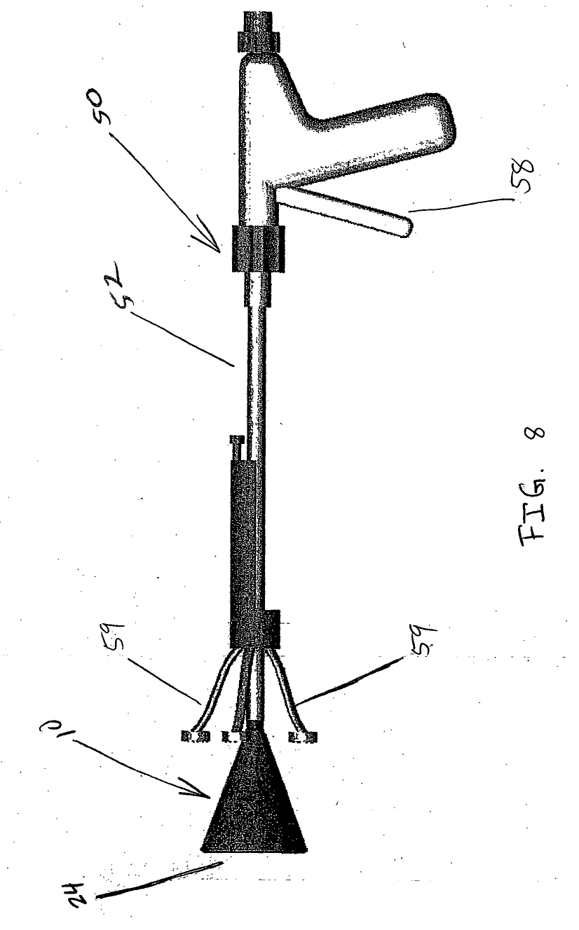

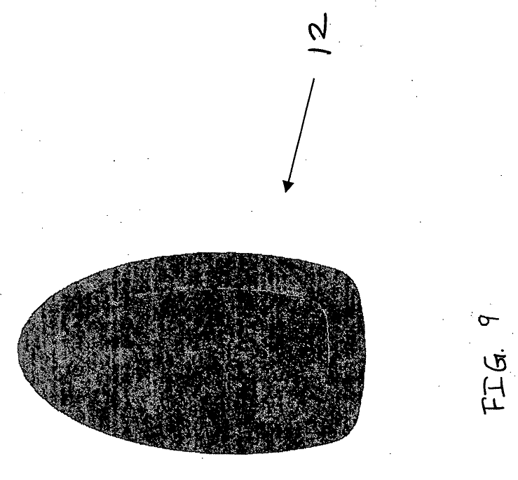

As shown in FIG. 7, the present inventive kit may further comprise a loading device 50 within which the shaping device 10 can be compressed. The loading device comprises a sheath 52 that comprises an inner shaft 54 and an outer shaft 56. The shaping device 10 self-expands once the inner shaft 54 element is advanced past the distal end of the outer shaft element 56, which removes the constraining force. The device may be capable of expanding to one, or more than one size (90, 110, and 130 cc, or small, medium, and large for example), by continuing to move the inner shaft 54 (with the expanding element) forward (relative to the outer shaft 56). The corresponding size of the expanding element may be indexed and referenced on the proximal end of the device. In addition, a detent or other design may be used to index the movement of the inner shaft 54 (and size of the expanding element). The movement may be controlled by a trigger mechanism, thumb slide, screw, combination or another suitable method.

In one embodiment, the inner shaft 54 comprises a tube or rod and may have a component molded or bonded onto the proximal end of the tube to act as a maximum travel stop when advancing the inner shaft 54 element forward. The inner shaft 54 may comprise a polymer, stainless steel, aluminum, superelastic/shape memory materials (such as nitinol), and/or any other suitable material. The inner shaft 54 may be fabricated using extrusion, casting, sintering, molding, machining, a combination of these, or any other suitable method or methods. The shaping device 10 may be attached to the inner shaft 54 by adhesives, soldering, sonic welding, spot welding, mechanical interference fit, any combination of these, or by another suitable attachment method or methods.

In one embodiment, the outer shaft 56 comprises a tube into which the inner shaft 54 is movably inserted. The inner diameter of the outer shaft 56 may be smooth and round, or may have longitudinal “riflings” or grooves. The outer shaft 56 may be connected to a knob or another control to allow rotational movement of the outer shaft 56. The outer shaft 56 may have one or more additional lumens to aid in advancing a diagnostic, therapeutic, or other device along the shaft. In one embodiment, the outer shaft 56 has a tip set at a 90° angle; however, in other embodiments the tip may be set at different angles.

The outer shaft 56 may be fabricated using extrusion, casting, sintering, molding, machining, any combination of those methods, or any other suitable method or methods. In one embodiment, the outer shaft element 56 is preferably made from a polymer, stainless steel or aluminum, however, it may comprise any other suitable material. In an alternate embodiment, the outer shaft 56 may comprise superelastic/shape memory materials (such as nitinol).

As described above, the shaping device 10 may include vacuum lumens or tubes. The inner diameter of the outer shaft 56 may have riflings or grooves that can help the shaping device 10, particularly if it has vacuum lumens or tubes, to slide into and out of the outer shaft 56. The inner shaft 54 may have one or more additional lumens to aid in advancing a diagnostic, therapeutic, or other device along the shaft. Referring to FIG. 8, the securing device 59 may alternately or additionally comprise an exterior organ vacuum, which may be fixedly or slidably attached to the exterior of a device used to aid in the placement or movement of the shaping device 10.

In an alternate embodiment, a ring, band, gasket or something similar may be located on the outside of the outer shaft element 56, moveable (by hand or stylet, for instance) up against the outside of the ventricular apex (or other desired location) to prevent slippage, or other in/out movement, while the expandable element is within the hollow organ.

In one embodiment, a proximal handle allows the inner shaft element 54 to be inserted into the inner diameter of the outer shaft element 56, and may include a means (such as a rotating friction mechanism) to prevent the inner shaft element 54 from moving. The proximal handle may also include controls to move the distal tip, end, or any other section of the outer shaft element (for the movable tip version of the device) similar to a wrist or elbow joint. In an alternate embodiment, the proximal handle may have additional features such as an automatic indexing feature for the inner shaft 54 movement, a vacuum, and/or fluid connectors, or lumens or pathways for a surgical instrument or tool. Fluid connectors may enable a user to infuse a gas (for example, CO2) or a liquid (for example, saline) from the proximal handle, through a tube or lumen, to the distal end of the device. The proximal handle may include a rotating device (or something similar) that can be used to compressively lock the position of the incising element or inner shaft element 54 (similar to a Touhy-Borst fitting).

In one embodiment, the proximal handle is made by using injection molding techniques and comprises polycarbonate. In other embodiments, the handle may be made from polyetheretherketone (PEEK), PVC, a combination of these, or of another suitable material or materials. The proximal handle may also be made using machining, casting, molding, a combination of these, or another suitable method or methods.

The shaping device 10 may also be deployed through a standard or modified endoscope. For a minimally invasive procedure, the endoscope may have an incising tip that can be used to make an incision in the akinetic tissue, ventricular apex, or any other desired location in the heart chamber wall. The endoscope can then be inserted through the incision, and the shaping device 10 may be inserted through the endoscope. The device could also be used for laparoscopic, robotically assisted, minimally invasive, open field and/or percutaneous procedures. The system may include a fixed or removable component at the distal tip to incise tissue as the device is being advanced into tissue.

In yet another embodiment, an RF or DC heating element (direct resistive element or ohmic tissue heating) on the outside of the cone (with or without temperature sensing—thermocouple/thermistor or other) may allow the shaping device 10 to be used to ablate the inside of a hollow organ, body cavity, and/or another location, for example an intra-uterine endometrial ablation (U.S. Pat. No. 5,865,801 to Houser is herein incorporated by reference).

Patch

The kit of this invention may further comprise a hemostatic patch 12 as described in co-pending U.S. patent application Ser. No. 10/183,396, filed on Jun. 28, 2002 and titled Patches and Collars for Medical Applications and Methods of Use, which is incorporated herein in its entirety by reference. As described in more detail below, the patch 12 shown in FIG. 9 is generally simple to use, inexpensive, and effective at providing hemostatic sealing. The patch 12 may be used for several different applications in the body, including, but not limited to, repair of the left ventricle or another heart chamber as described above.

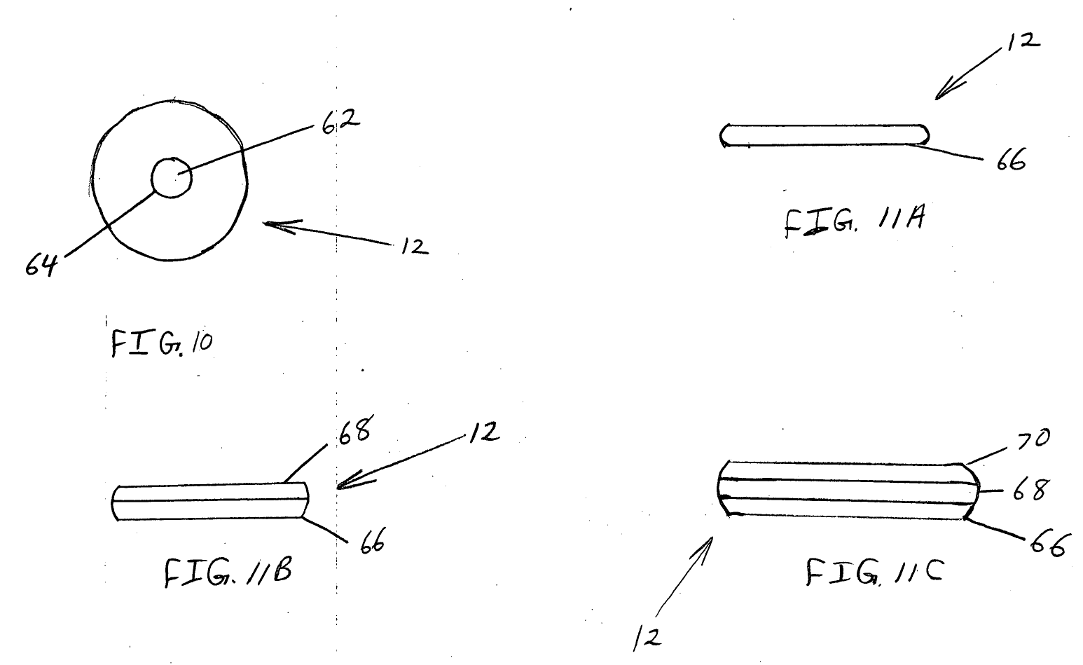

Referring to FIG. 10, the patch 12 may have an elongated shape, which can aid in achieving the desired result of a heart chamber that is elongated rather than spherical. In addition, the patch 12 may be either completely or partially flat, convex or concave. The convexity or concavity can further aid in achieving a heart chamber of the appropriate size and shape by guiding the curvature of the heart chamber wall.

In use, the hemostatic patch 12 is designed to be of a sufficient size, shape, strength flexibility, and thickness for the purpose of closing a heart chamber opening or, for example, to provide hemostasis, particularly at a heart chamber access puncture site, or for other suitable purposes. The properties of the patch 12, e.g., rigidity, flexibility, tissue closure compressive force, may be modified by varying one or more of the geometry, thickness, material, components, processing, or other characteristic of the patch 12.

Referring to FIG. 10, one embodiment of the patch 12 includes an opening 62 and a slit 64. The patch 12 may be passed over a device that is inserted into a hollow body cavity to provide a temporary or permanent seal. The cross section can be consistent or tapered. A deployment device 60 with a grasping and releasing distal end, described below, may be used to position and deploy this version of the patch 12.

The patch 12 may be utilized during an open field or minimally invasive surgical procedure. It may also be deployed through a standard or modified endoscope. The shaping device 12 may also be used for laparoscopic, robotically assisted, percutaneous, or catheter based procedures. The shaping device 12 may be compressible and re-expandable to allow compression during insertion and withdrawal, and re-expansion once inserted into the organ. The ability to compress the shaping device 12 into a reduced cross section profile facilitates insertion and removal.

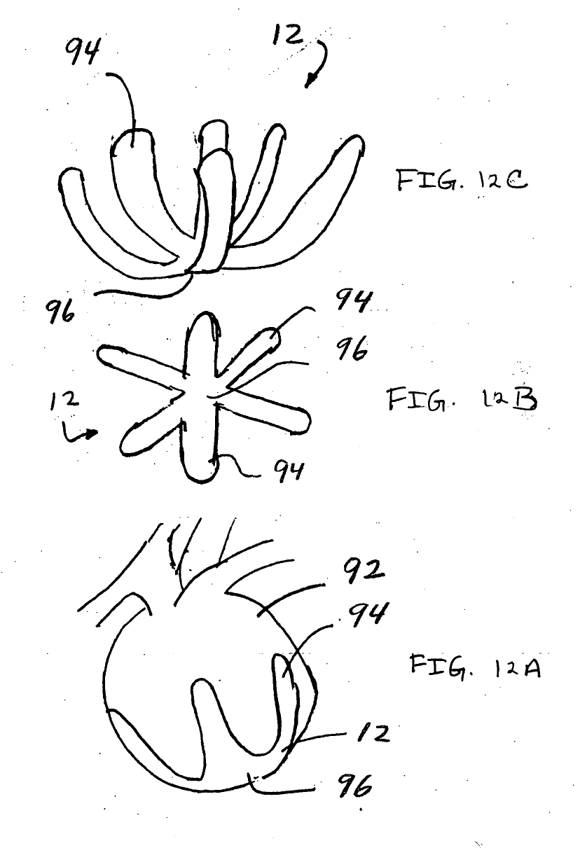

Embodiments of the patch 12 may comprise a wide range of different shapes and sizes. Alternate embodiments having different shapes and sizes are described in detail in a co-pending U.S. patent application Ser. No. 10/183,396, incorporated herein by reference in its entirety. As shown in FIGS. 11A-11C, the patch 12 may have a single layer 66, dual layer, 66 and 68, or multiple layers, for example, having three layers 66, 68 and 70.

As described in more detail below, the patch 12 may be fabricated with or without a superelastic/shape memory component or other reinforcement that is capable of compression. These components or reinforcements can be one of the layers illustrated in FIGS. 11A-11C, discussed above. The thickness of the patch 12 may be the same throughout or vary as desired for a particular application.

The tissue contacting surface may be flat, smooth, irregular, woven, or include dimples or protrusions. These surface configuration can be selected for several purposes including bonding, securing, tissue growth, etc. The patch 12 may also have one or more holes, pores, grooves, slots, or openings that pass partially or completely through the patch 12.

The tissue contacting surface of the hemostatic patch 12 may have a coating or layer of a biocompatible contact adhesive, or other material to bond or secure the patch 12 to the vessel or heart chamber to better seal the puncture site or opening. For example, the adhesive layer can be layer 66 of FIGS. 11B and 11C. The bonding materials can be added during the manufacturing process or just prior to use. The bonding materials could be in the form of a liquid, semi solid, or solid. Suitable bonding materials include gels, foams and micro-porous meshes. Suitable adhesives include acrylates, epoxies, fibrin-based adhesives, UV light activated adhesives and/or heat activated adhesives and other specialized adhesives. The adhesive can be selected to bond on initial contact, or after a longer period to allow repositioning if desired. One effective adhesive is a crystalline polymer that changes from a non-tacky crystalline state to an adhesive gel state when the temperature is raised from room temperature to body temperature. Such material is available under the trade name Intillemer™ adhesive, available from Landec Corp. Composites and combinations of these materials also can be used.

Alternately, the tissue contacting surface of the patch 12 may include barbs 71, or other protrusions, to secure the patch 12 to the vessel or heart chamber. The barbs 71 can be oriented to retain the device against the heart. For example, the barbs 71 can extend directly from the patch 12 or at an angle from the patch 12. As described in detail in copending U.S. patent application Ser. No. 10/183,396, incorporated herein by reference in its entirety, the patch 12 and barbs 71 can be electrically connected to a power source and controller to apply or supply heat to the barbs, causing the barbs 71 to heat the tissue through which they pass for securing or any other purpose.

The hemostatic patch 12 may be partially or completely fabricated from many different types of biocompatible materials, including expanded polytetrafluoroethylene (“ePTFE”), polyester, woven Dacron®, polyurethane, silicone, a composite material, or a combination of these or other suitable materials. Some polymer materials could be irradiated in a desired geometry, for the shape to be “set” into that position. This setting is advantageous if it is helpful to provide a particular profile to the heart chamber. For example it may be helpful to provide a compressive force to the heart chamber once the patch 12 is deployed around the heart chamber. A similar process using heat instead of radiation can be used to anneal the polymer and then cool the polymer into a particular shape.

The patch 12 also may be partially or completely made from many different types of biodegradable/bioabsorbable materials, including modified starches, gelatins, cellulose, collagen, fibrin, fibrinogen, elastin or other connective proteins or natural materials, polymers or copolymers such as polylactide [poly-L-lactide (PLLA), poly-Dlactide (PDLA)], polyglycolide, polydioxanone, polycaprolactone, polygluconate, polylactic acid (PLA), polylactic acid-polyethylene oxide copolymers, poly(hydroxybutyrate), polyanhydride, polyphosphoester, poly(amino acids), poly(alphahydroxy acid) or related copolymers of these materials as well as composites and combinations thereof and combinations of other biodegradable/bioabsorbable materials. The patch 12 may also be fabricated to include a radiopaque material, such as barium sulfate, bismuth trioxide, tantalum or other radiopaque. The radiopaque material can be added to the device itself, the reinforcement structure, or the bonding material.

Additionally, the patch 12 may be partially or completely fabricated from materials that swell, or expand when they are exposed to a fluid, such as blood, other body fluid, or other fluid that can be applied in use. These materials include hydrophilic gels (hydrogels), foams, gelatins, regenerated cellulose, polyethylene vinyl acetate (PEVA), as well as composites and combinations thereof and combinations of other biocompatible swellable or expandable materials.

The hemostatic patch 12 may be fabricated using several methods and processes including extrusion, molding (e.g., injection molding or other known molding techniques), casting, sintering, laminating, weaving, knitting, dip coating, spraying, as well as combinations of these and other methods and processes. The patch material may be formed into various geometries by die cutting, heat forming, laser cutting, or other similar methods.

In one embodiment, the hemostatic patch 12 may be configured to include a metallic component, such as a wire, rod, tube, coil, sheet, strip, band, in the middle, outer region, interior, or one or more sides of the patch 12. The metallic material may be a superelastic/shape memory alloy such as nitinol. The superelasticity can allow for greatly improved collapsibility during insertion, and can allow the patch 12 to return to its intended original shape when removed from the deployment device (catheter, endoscope, etc.). The high degree of flexibility is also more compatible with the stiffness of the engaged heart chamber wall.

In one embodiment, the edge of the patch 12 may have a collar or rim made of a superelastic/shape memory material, an elastic combination of materials, or suitable elastic materials. In another embodiment, a superelastic/shape memory layer may be located along the full, or a partial, length or width of the patch 12. Referring to the layers illustrated in FIGS. 11A-11C, a shape memory alloy 72 may be positioned within an inner layer 68 and surrounded by an upper layer 70 of a biocompatible polymer and a lower layer 66 of a biocompatible polymer. The polymer may be any of the polymers described herein, such as Dacron® or ePTFE.

Superelastic/shape memory materials in tubular, rectangular, wire, braid, flat, or round form, or any combination of these or other structures also can be used in the design of the patch device, to assist with grasping, contacting, bringing tissue together, sealing, or other desired function. Superelastic or shape memory materials comprise any material that is inherently resistant to permanent deformation and/or is processed to be resistant to permanent deformation. These materials can be formed or set in a geometry matching the desired geometry of the vessel or heart chamber and can aid in urging the vessel or heart chamber into that desired geometry. Certain shape memory materials, when exposed to normal body temperature (37° C. ), will return to a set shape thereby applying pressure to the vessel or heart chamber. Similarly, certain superelastic materials can be initially deformed or deflected during deployment and then can recover a set shape.

When thermally forming the superelastic component layer, the superelastic material(s), which have been previously cut into the desired pattern and/or length, are stressed into the desired resting configuration over a mandrel or other forming fixture having the desired resting shape. The resting shape of the patch 12 depends on the size of the heart chamber or other location in which the patch 12 is intended to be used. After being stressed into the resting configuration, the material is heated to approximately between 300 and 650 degrees Celsius for a period of time, which is typically approximately between 30 seconds and 30 minutes. Once the volume of superelastic material reaches the desired temperature, the superelastic material is quenched by inserting it into chilled water or other fluid, or otherwise allowing the material to return to ambient temperature. In this manner, the superelastic component layer(s) are fabricated into their resting configuration.

It is important to understand basic terminology when describing metals with elastic, superelastic, or shape memory behavior. Elasticity is the ability of the metal, under a bending load, for example, to deflect (i.e., strain) and not take a permanent “set” when the load (i.e., stress) is removed. Common elastic metals can strain to about two percent before they set. Superelastic metals are unique in that they can withstand a larger strain before taking a set. Some superelastic materials can withstand up to about 5% or 6% strain before taking a set, other superelastic materials can even withstand an impressive 10% strain without taking a set.

In some superelastic/shape memory materials, the higher elasticity is attributed to a “stress-induced” phase change within the metal to allow it to withstand such dramatic levels of strain. Depending on the composition of the metal, the temperature that allows such a phase change can vary. And if the metal is “set” at one temperature, and then the temperature is changed, the metal can return to an “unset” shape. Then, upon returning to the previous “set” temperature, the shape changes back. This is a “shape-memory” effect due to the change in temperature changing the phase within the metal.

Elasticity is a key feature of superelastic materials. When a metal is loaded (i.e., stressed) and undergoes, for example, bending, it may deflect (i.e., strain) in a “spring” fashion and may tend to return to its original shape when the load is removed, or it may tend to “set” and stay in a bent condition. This ability to return to the original shape is a measure of the elasticity or “resilience” of the metal. This ability for a metal to be resilient is desirable for such things as springs, shock absorbing devices, and even wire for orthodontic braces where the ability, to deflect, but not deform (i.e., set) is important to maintain an applied force.

If, under a bending load, the metal takes a set, it is said to have plastically (versus elastically) deformed. This is because the imposed stress, produced by the bending load, has exceeded the “elastic limit” of the metal. If the applied load increases past the elastic limit of the metal, it will produce more plasticity and can eventually break. The higher the elastic limit of the metal, the more elastic it is. “Good” elastic metals can accommodate up to about two percent strain prior to taking a set. However, this is not the only factor governing “elasticity.”

Another factor that determines the ability of a metal to deflect to a given, desired amount, but not take a set, is the “elastic modulus,” or often called the modulus of elasticity. The modulus of the metal is an inherent property. Steels, for example, have a relatively high modulus (30 msi) while the more flexible aluminum has a lower modulus of about 10 msi. The modulus for titanium alloys is generally between 12 and 15 msi.

Resilience is the overall measure of elasticity or “spring-back ability” of a metal. The ratio of the yield strength divided by the modulus of the metal is the resilience. Although it is one thing for a metal to be resilient, it must also have sufficient strength for the intended service conditions.

The most common superelastic metal, used in many commercial applications, is an alloy comprised of about equal parts of nickel (Ni) and titanium (Ti), and has a trade name of “nitinol.” It is also referred to as “NiTi.” By slightly varying the ratios of the nickel and titanium in nitinol, the stability of the internal phases in the metal can be changed. Basically, there are two phases: (1) an “austenite” phase and (2) a lower temperature, “martensite” phase. In the malleable martensitic state, the alloy can be easily deformed (set). Then upon heating back to the austenitic temperature, the alloy will freely recover back to its original shape. Then if cooled back to the martensitic state, the deformed shape reforms.

In general, the Ni-to-Ti ratio in the nitinol is selected so that the stress-induced martensite forms at ambient temperatures for the case of superelastic brace and support devices, which are used in ambient conditions. The specific composition can be selected to result in the desired temperature for the formation of the martensite phase (Ms) and the lower temperature (Mf) at which this transformation finishes. Both the Ms and Mf temperatures are below the temperature at which the austenite phase is stable (As and Af).

By manipulating the composition of nitinol, a variety of stress-induced superelastic properties can result, and over a desired, predetermined service temperature range. This allows the metal to behave in a “shape-memory” or “shape recovery” fashion. In this regard, the metal is “set” to a predetermined, desired shape at one temperature when in a martensitic condition and returns to the original shape when the temperature is returned to the austenitic temperature.

Based on the background information provided above, it can be seen that if the nitinol material requires an exceptionally tight bend, and one that would normally exceed the elastic limit of the material and thus permanently deform it, a bend can be placed in the device and the device annealed to relieve bending stresses within the device. Following this first bend, the device can be bent further to produce an even sharper bend and then re-annealed to alleviate the stress from this additional bending. This process can be repeated to attain the desired, sharp bend or radii that would otherwise permanently deform the device if the bend were attempted in a single bending event. The process for recovery from the position of the most recent bend is then performed as described above.

Although the example of nitinol, discussed above, is, by far the most popular of the superelastic metals, other alloys can also exhibit superelastic or shape memory behavior. Some examples of superelastic materials include the following:
Copper-40 at % Zinc
Copper-14 wt % Aluminum-4 wt % Nickel
Iron-32 wt % Manganese-6 wt % Silicon
Gold-5 to 50 at % Cadmium
Nickel-36 to 38 at % Aluminum
Iron-25 at % Platinum
Titanium-40 at % Nickel-10 at % Copper
Manganese-5 to 35 at % Copper
Titanium-49 to 51 at % Nickel (nitinol).
   The patch 12 may comprise any of these or other superelastic/shape memory materials as well.

nitinol, because of the large amount of titanium in the composition, has been the only FDA approved superelastic/shape memory alloy for medical implant devices. The corrosion resistance of nitinol is superior to that of commonly used 3161 stainless steel, and, if surface oxidized or passivated carefully, can reach corrosion resistance comparable to the most popular titanium implant alloy, Ti6A14V. Similarly, if desired the metal piece can be electropolished to improve its biocompatibility and blood compatibility. Biocompatibility studies have routinely showed nitinol as a metal with suitable biocompatibility for medical device applications.

In summary, there are various ways of describing elasticity, but the main criterion is the ability of the metal to return to its initial, pre-loaded shape. Some metals can only deflect a couple percent and remain elastic while others, such as superelastic nitinol, can deflect much more. nitinol is also biocompatible and corrosion resistant. This unique combination of properties may allow a device made of nitinol, such as a patch 12, to be fully collapsed within a deployment tool and be subsequently released at a particular site within, in between, or on the surface of the desired location to form its intended service shape.

Materials other than superelastic/shape memory alloys may be used in place of superelastic/shape memory alloys provided they can be elastically deformed within the temperature, stress, and strain parameters required to maximize the elastic restoring force thereby enabling the patch 12 to recover to a specific diameter and/or geometry once deployed inside, over, or on top of the vessel or heart chamber or other location. As used in this application, the terms “shape memory material” and “superelastic material” refer to any material that can be elastically deformed within the appropriate temperature, stress and strain parameters. Some examples of such materials include shape memory alloys, spring stainless steel 17-7 PH, other spring metal alloys such as Elgiloy™, Inconel™, superelastic polymers, etc.

Any metal or metal alloy, such as a superelastic/shape memory alloy that comes in contact with blood and/or tissue can be electropolished. Electropolishing may reduce platelet adhesion causing thrombosis, and encourage endothelization of the exposed metallic areas. Electropolishing also beneficially removes or reduces flash and other artifacts from the fabrication of the device.

The hemostatic patch 12 also may have the ability, once positioned at the desired location, to compress the heart chamber wall for increased securement and sealing. In this embodiment, the patch 12 may be used as a clamping or compression device. This can be accomplished by making the patch 12 completely or partially from a very elastic material that is stretched while being secured to the heart chamber wall and allowed to recover after being secured to the heart chamber wall (i.e., when the deployment device is separated from the patch). The elastic material may include or be a layer of a superelastic/shape memory material to assist with the closure or reinforcement. The recovery of the elastic material may be configured to cause the ends of the puncture site to plicate, or be brought together.

In one embodiment illustrated in FIGS. 12A-12C, one or more of the patches may be placed on the outside of the heart 92 near the left ventricle to treat congestive heart failure (“CHF”) by preventing, delaying, or limiting remodeling and to assist the left ventricle to decompress during systole based on the superelastic/shape memory properties of the metal alloy within the patch 12. In general, the device is placed to constrain the outside of the heart 92 without significantly interfering with the normal movement or function of the heart to prevent remodeling of the heart tissue. In this manner, the device assists the ventricular contraction of the heart 92 by providing a device that, when deflected outward, will tend to return to the as-annealed configuration of the superelastic/shape memory reinforcing member contained on, inside, or outside the device. The device can be fabricated from single or multiple strips or bands. To be as atraumatic as possible, the strips or bands can be fabricated with rounded ends.

Referring to FIGS. 12A-12C, in one embodiment the patch 12 may be configured in a generally star pattern that includes arms 94 and a base 96. The device 12 is positioned on a heart 92 in a centered manner on the bottom apex of the heart. Of course, the device 12 may be centered on other locations of the heart 92, such as the left ventricle and/or the right ventricle, such that the device resists remodeling while nonetheless assisting the heart to attain systole.

The device 12 may include an atraumatic tissue contacting surface (e.g., such as ePTFE or woven Dacron®) that may optionally be provided with an adhesive on or near the tissue contacting surface. The device may include one or more layers (e.g., in the form of a strip, band, wire, tube, rod, mesh, etc.) of superelastic/shape memory material, or other reinforcing material as previously disclosed herein. The superelastic/shape memory material may be annealed in any configuration as required or desired such that when deflected or forced from its annealed configuration, it will have a tendency to return to its annealed configuration. Multiple strips or strip ends may be independent, or attached to one another or a combination of both. The ends may be attached to each other by using a mesh, single or multiple strips, bands, wires, and/or tubes. The attachment(s) may be elastic, semi elastic, rigid, or have a combination of these properties. The attachment may be made by using any of the methods described herein or using any commonly known technique. The rigidity, flexibility, closure, and/or compressive force of the device 12 may be modified by varying, for example, the device's geometry, thickness, material, component(s), or processing.

Along with, or in place of, adhesive used to adhere the patch 12 to the heart chamber, heat can be used as for the deployment/securing/bonding/healing process of the patch 12. The heat can be used to recover the patch 12, activate and cause a hemostatic material to flow to the puncture site and or around the patch 12, activate the shape memory/superelastic alloy component layer, activate a therapeutic substance, assist in sealing, accelerate healing, or a combination of these or other effects.

Direct resistive element heating or ohmic tissue heating can be used to provide the heat. A biocompatible electrode material (e.g., gold, platinum, a combination of these or other suitable material) can be mixed with the patch base material as a powder during compounding. Alternatively, strips or wires can be added onto any surface, or any layer of the patch 12. Additionally, sputter coating, ion beam deposition, spraying, or adhesive bonding can be used to produce an electrode, which can then be connected to a suitable wire conductor. For ohmic tissue heating, one end of a conductor could be connected to an RF power source, with the other end attached, either directly or through a cable, to the electrode. Another conductor could be connected at one end to a ground pad placed on the patient's body with the other end connected to the power source. For direct resistive element heating, both conductors from the power source would be connected to the electrode. Once the puncture site has been sealed, the physician twists, cuts, or otherwise removes the conductor attached to the patch 12. Alternatively, a special tip can be placed over a standard electro surgical tool (e.g., Bovie) to insert through the skin and make contact with the patch 12 and/or tissue.

In another embodiment, the patch 12 may further comprise one or more therapeutic agents that positively affect healing at the site where the device is deployed, either incorporated into the structure forming the device, or incorporated into a coating, or both. Such therapeutic agents may include, but are not limited to, antithrombotics (such as anticoagulants), antimitogens, antimitotoxins, antisense oligonucleotides, gene-therapy solutions, nitric oxide, and growth factors and inhibitors. Direct thrombin inhibitors that may be beneficial include Hirudin, Hirugen, Hvrulog, PPACK (D-phenylalanyl-L-propyl-Larginine chloromethyl ketone), Argatreban, and D-FPRCH.sub.2 CI (D-phenylalanyl-Lpropyl-L-arginyl chloromethyl ketone), indirect thrombin inhibitors include Heparin and Warfarin. Alternatively, a clot promoter may be used, such as protamine sulphate or calcium hydroxide.

The patch 12 may be placed on or inside a heart chamber by hand or by using a deployment device. Different embodiments of the deployment device are described in detail in detail in copending U.S. patent application Ser. No. 10/183,396, incorporated herein by reference in its entirety.

Shaping Suture

Certain surgical methods seek to attain asymmetric morphologies for geometric reshaping of dysfunctional organs or structures, in order to make them conform to more optimal function. Circular patch-plastics such as the Dor Procedure described above, while easy to construct, exert naturally circular forces of the cinching down of a classic purse-string suture, and tend to create a spherical reshaping by virtue of the fact that the dimensions are altered equally in all directions. In applications where an eccentric patch-plasty is desirable, a device that can exert unequal forces on the structure would allow the ease of purse-string suture placement, while at the same time, distribution of the altering forces differentially.

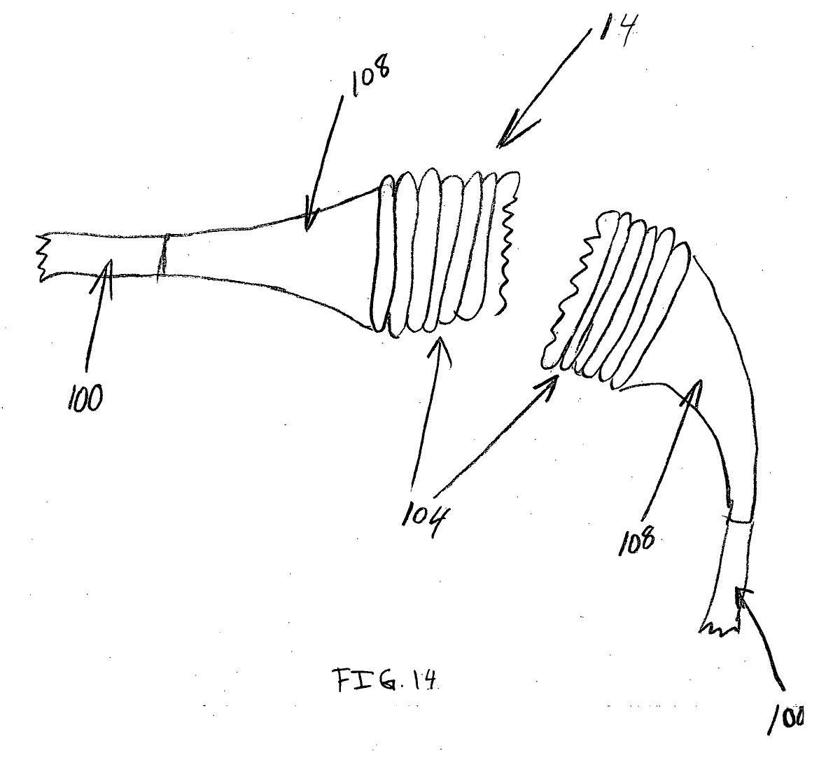

The kit of the present invention may also comprise a shaping suture 14. In one embodiment, the shaping suture 14 of this invention, illustrated in FIG. 13, comprises an elongate filament comprising a suture element 100 with needles 102 at either end, with an annealed 104 portion positioned generally centrally. When properly placed and deployed, the shaping suture 14, placed as a circular purse-string, would form a non-circular reduction, such as one having a tear-dropped or oval shape. This device would selectively allow decrease in one dimension while having a lesser impact on another dimension. Thus, this aspect of the invention comprises a device 14 that can be placed in tissue like a standard, double-armed, purse-string suture, but when properly cinched down in the tissue, takes on a distinctly non-circular shape, such as a conical, ovoid, or elliptical one, even if applied to a circular defect.

As discussed above, in selected cases of congestive heart failure, benefit has resulted from an operation where the large, spherical configuration of the failed ventricle 20 is remodeled using an endocardial circular patch-plasty, (or “Dor Procedure”). The ventricle 20 is incised through an area of dysfunctional scar, after which a purse-string suture (the “Fontan stitch”) is placed along the margin between viable and scar tissue.

Instead of a circular purse-string, which when tightened would only create a smaller sphere out of the ventricle, this device 14 would have the effect of decreasing the ventricular wall-size in the short axis (cross-sectional dimension), while leaving the long axis only slightly impacted. This would result in a conical (more normal and therefore more physiologic) reshaping of a previously spherical chamber.

This impact can be enhanced by a tear-shaped patch 12 as described above, which will be attached to the endocardial surface at the margin of the purse-string. The patch 12 will be covered again with excess scar tissue excluded from the chamber by the purse-string and the patch 12.

In one embodiment, this device 14 may have built-in, profound short axis reduction, with controllably less long axis shortening. The device may be designed in a relevant range of sizes. The optimal size and shape of the ventricle can be predetermined through a process that can allow selection of the ideal device, implanted over a pre-shaped shaping device 10. This can allow a standardized surgical procedure that can imprint pre-planned ideal dimensions on a reconstructed ventricle with less operator variation.

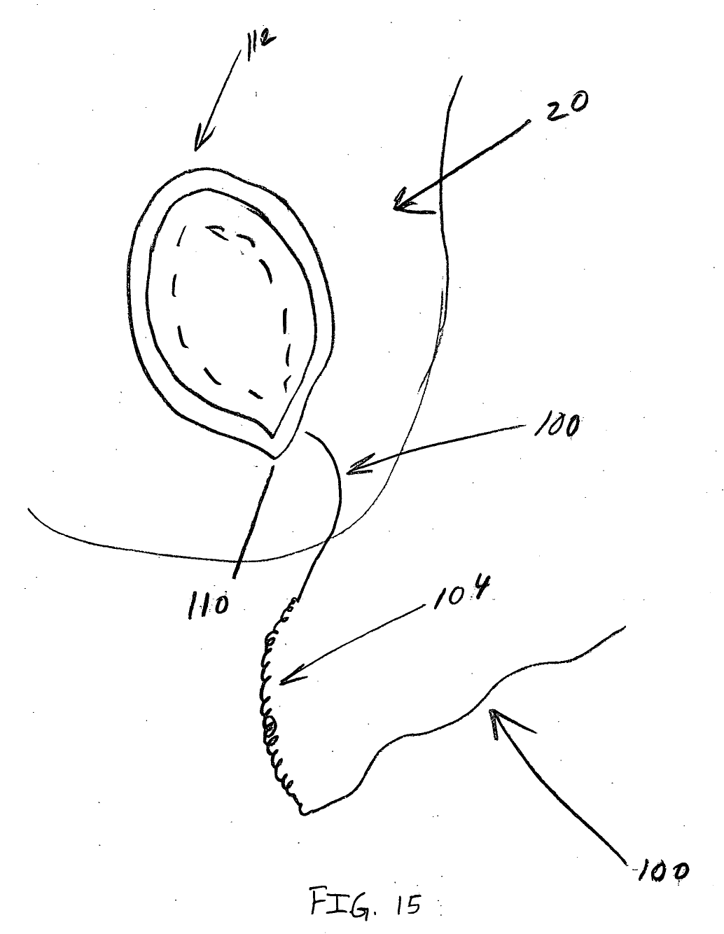

In one embodiment, illustrated in FIGS. 13 and 14, the suture element 100 comprises a standard 30 inch 2-0, double-armed polypropylene suture with SH or SH-1 needles on either end. In this embodiment, the annealed portion 104 comprises a 6-inch (variable) nitinol member occupying the middle of the length of the shaping suture 14. In its relaxed state, it can be flexible, such that it can readily conform to the tissue as the purse-string is being placed.

In one embodiment, the device may have a funnel-shaped, tapering transitional segment which can allow the greater diameter of the annealed portion to slide easily into the track created by the suture as it weaves through the scar-tissue. The surface of the annealed portion may be lubricated to enhance its slipping through the tissue without friction or cutting. The tapering section 108 may be molded, or bonded by any conventional technique, including swaging, crimping, sonic welding, soldering, heat forming, adhesives, solvent bonding, and combinations thereof. The material of the suture element 100 may be bonded to the needle 102 and/or annealed section 104 externally, internally, or by any combination thereof.

The annealed portion 104 may comprise any shape memory material. For example, shape memory alloys, polymers, spring stainless steel, 17-7 PH, other spring metal alloys such as Elgiloy™, Inconel™, superelastic polymers, combination or other suitable materials may be used. The attachments of the two ends of the annealed portion 104 may be crimped, clipped, bonded with an adhesive, heated, or otherwise connected in a secure and reliable manner. The two ends may be capped, or otherwise covered to reduce the potential for the ends to perforate or abrade adjacent tissue. The cap may further include a sealing adhesive or potting compound, bonding the cap to the ends of the annealed portion of the suture device 14. The demands of the annealed portion may necessitate that it be applied in multiple pieces, each of which will be connected to the next by an appropriate technique as mentioned above, or any suitable method. Shapes other than tear-dropped or oval may be desired and therefore any annealable shape may be applied to the device if it is deemed useful.

Superelastic, shape-memory materials (nitinol, for example) may be subjected to an annealing process, as known to those skilled in the art, typically by constraining the component in a desired configuration, annealing at temperatures typically ranging from 300° C. to 600° C., for typically between 30 seconds and 30 minutes, quenching with ice water (or other suitable method) and repeating as desired to impart a desired resting configuration.

The suture device 14 may also comprise one or more sections that are wires, rods, tubes, coils, sheets, strips, bands, or any combination or other suitable geometry. The device 14 may be of any suitable length and may have any suitable needle size or shape. The suture element 100 may be monofilament or braided, coated or uncoated, absorbable or non-absorbable. It may be of any appropriate thickness, and may have different strengths for different sized nitinol nooses 104. The transition elements 108 may be long or very short.

In alternate embodiments, the shaping suture 14 may comprise a malleable or deformable material, typically metal, or metal alloy, which is capable of non-elastic deformation. Exemplary malleable materials include stainless steel and the like. The ends of the annealed section 104 may utilize ratchets, detents, or other interlocking components to permit closure and securing.

In certain embodiments, the annealed section 104 may be separated from the suture element 100 and/or the needle 102 by cutting, cleating, or any other method or process. In some embodiments, the suture element 100 and or annealed section 104 may be detached or cut to length simply by bending the two elements to an acute angle. In other embodiments, the transition region 108 between the suture material and annealed section and/or needle may include a weakened section, such as a notch, hole, cut-out, groove, reduced cross-section, or otherwise weakened area, to permit the rapid detachment by bending, cutting or other action.

The shaping suture device 14 may be treated in a variety of conventional or unconventional ways such as coating, jacketing, over molding, dipping, spraying, casting, or combinations thereof. Such layers, coatings, or other materials may be intended to provide a softer contact area, adhesives, provide a drug elution layer, or the like.

The shaping suture 14 may comprise more than one annealed section 104. For example, there may be two or more sections of annealed material, with a piece of standard suture material (2 to 4 cm or other) connected between them. Once sutured into tissue, the top (standard suture) may be semicircular (or other shape), while the sides (annealed material sections) may be straight (or another shape, different that what it would be if only standard suture was used). In one embodiment, one or both ends of the device may have a loop, that the second end may be inserted into, tensioned and secured for joining the ends together.

Preferably, the purse-string suture can be started at any point along the loop, but should exit at the site 110 closest to the apex of the left ventricle 20, since the sharp tip of the tear-shape will form at the exit site 110, while the rounder end 112 forms 180 degrees from the exit site, (and in this example, closest to the base of the heart). The long axis will have a lesser decrease in dimension, depending on how the nitinol or other material is annealed.

The practitioner may simply apply the nitinol suture by hand or may use a deployment device. One such deployment device comprises a sheath with a handle and a stylet. The practitioner can back-load the suture into the sheath, and then the practitioner can advance the suture using the stylet. Although this particular deployment device is described by way of example, any other suitable deployment device may be used.

In one embodiment shown in FIG. 16, the device may be tightened, possibly over a pledget, possibly on the outside of the ventricle 20 wall rather than the inside, possible with the use of a strain gauge to optimize tension, and the most proximal, exposed segments of the nitinol will be attached, with a crimping tool and device 114 or other suitable securing device and method.

With the cinching and fixation of the nitinol noose, the desired, previously annealed shape will be applied to the defect in the endocardium demarcated inside the purse-string. The patch 12 can then be used to cover the defect. The size of the patch 12 may be pre-conformed both to the needs of the individual patient and to the nitinol stitch.

Because the compliance of the myocardium, and therefore the final post deployment size, can be variable, final sizing of the patch 12 can be gauged by a series of sizers, available within the relevant range. This can ensure accuracy and standardization of such a procedure.

This device and its driving concepts could be applied in any reconstruction situation where a shape other than round is desired or where a minimum smallest size is desired. For example, bowel anastomoses might be improved upon it an annealed circumference stitch would create a supported size connection that would remain round with a circumference that could not be deformed smaller than a given size. It could also serve as a template for procedures such as gastric stapling, removing variability from the sizing of the restructured pouch. The nitinol may serve as a stent for collapsible structures such as the bronchus, forcing roundness in a compressible hollow structure. Integrated Sizer/Shaper and Patch

In another embodiment, illustrated in FIG. 17, the kit of the present invention may include an integrated device 120 comprising a shaping device 10 and a patch 12. As illustrated in FIG. 17, the patch 12 may be attached to the shaping device 10 through a removable stitch 122. In other embodiments, however, a temporary adhesive, clips, staples or any other suitable means of attachment may be used. A temporary method of attachment can allow the patch 12 to be detached from the shaping device 10 at an appropriate time during the procedure. If a stitch 122 is used, for example, it may be cut when it is desired to detach the patch 12 from the shaping device 10.

After the integrated device has been positioned within the heart chamber, the patch 12 may be attached to the tissue using a suture, barbs, protrusions, or any other appropriate device. The shaping device 10 may be detached from the patch 12 and may be removed once it is no longer needed within the heart chamber. The removal of the shaping device 10 may be facilitated with an initial temporary attachment to the tissue, which can later be converted to a permanent fixation.

As illustrated in FIG. 17, the integrated device 120 may comprise one or more shaping sutures 14 that can be used to attach the patch to the tissue. The one or more shaping sutures 14, may be attached to a rim on the patch 12, which may comprise shape memory material. In one embodiment, the shaping sutures 14 and the rim on the patch 12 comprise nitinol; however, any appropriate material may be used.

In certain embodiments, the patch 12 may constitute part of the wall 44 of the shaper 10. That is, it may be that when the patch 12 is removed the a portion of the shaper wall 44 may be absent. The patch 12 and the shaper 10 may also be partially or completely compressed. In that embodiment, the attached patch 12 and shaper 10, may be folded, as an umbrella, and may be passively or actively expanded once inside the ventricle.

Patch Sizing Template

Referring to FIGS. 18 and 18A, one embodiment of the inventive kit may further comprise a template device 130 for sizing the patch 12. In one embodiment, the template device comprises a handle member 132 and a template member 134. The template member 134 may be removably connected to the handle 1342 such that different template members 134 can be used with one handle 132 and different handles 132 with one template member 134.

In one embodiment, the template device 130 is configured such that a physician can place the template member 134 over or inside an area to which the patch 12 may be applied. In this embodiment, the template member 134 comprises a material that is translucent enough that when looking at the area through the template member 134, a practitioner can identify the hole to be sealed with the patch 12. The practitioner can then either trace the size and shape of an appropriate patch 12 onto the template member 134 using a marker or other marking device, or can trim the template member 134 down to the appropriate size and shape. The practitioner next may remove the template member 134 and use it to trim the patch 12 to the appropriate size and shape.

In one embodiment, the template device 130 may comprise silicone, glass, rubber, metal, any polymer, polyurethane, polyethylene, polypropylene, however, any other suitable material may be used. The template member 134 may be flat, concave, convex, or conical as desired by the physician. In addition, the template member 134, may comprise one or more grid marks. The grid marks, if used, may be a predetermined locations to aid in sizing the patch 12. The grid marks may be molded into the device, printed onto the device, or affixed to the device through any other suitable method.

Referring now to FIG. 19, in another embodiment 136, the template member 138 may be generally cone shaped or pyramid shaped such that the template member 138 has a larger cross sectional area at its proximal end 140 than at its distal end 142. The cross sectional shape of the template member 138 may be circular, oval, square, triangular, or any other appropriate shape. In this embodiment, the increase in the cross sectional area of the template member 138 is stepped rather than constant. Thus, the template member 138 has one or more steps 146 where the cross sectional area of the template member 138 increases. The practitioner can measure the size of the patch 12 by inserting the distal end 142 of the template member 138 into the incision and continuing to insert the template member 138 deeper into the incision until it closes the incision. At that point, the practitioner can determine which step or steps 146 of the template member 138 are within the incision and can size the patch 12 based on the cross sectional area of that step of the template member 138. In another alternate embodiment, template members of different sizes may be compared with the hole to determine the correct size for the patch 12.

Determining Optimal Post-Procedure Size and Shape

The kit of this invention may also include a system to monitor a congestive heart failure patient and to customize treatment using Magnetic Resonance Imaging (“MRI”), PET Scan, Echo, ultrasound, and/or other methods. The system may allow a physician to determine the current condition of the ventricle or any other hollow body cavity, as well as a more optimum size and shape for the ventricle or other hollow body cavity. It may also be used to produce custom versions of devices such as a shaping device 10, patch 12, or suture 14 to treat congestive heart failure. In addition, it allows a unique follow-up treatment where a patient can be monitored to assess long term cardiac function and overall health status. This system can help to optimize treatment by enabling a practitioner to treat a patient earlier in the disease process. That can give patients a longer life through treating heart failure earlier and helping to prevent the heart from growing in size as typically occurs with heart failure patients.

The system may be accessible through the Internet and may allow image storage and access by various authorized users remote from the site and each other. This can aid in collaboration on potential treatment/management options. It can also help to standardize assessment, planning, timing, and conduct of surgical (or other) treatment of the specified disease process. The system may incorporate firewalls, encryption, or other types of security to allow certain aspects of the files to be viewed only by authorized participants while others may be seen by unrestricted users for the sake of recruitment and public education.

The system may also include a software program that allows a designated person or persons to manipulate the images to sculpt a more optimal configuration. This aspect allows an abnormal cardiac chamber to be redesigned in a virtual realm in order to assess plausibility of an actual (surgical) restoration. The user may also be able to store and compare serial images over time to enable timing and appropriateness of intervention. A data gathering/registry system can collate data from all files to create a database to obtain accurate outcomes information.

In one embodiment, this system comprises a web-based site that stores data on a designated computer or other electronic storage system for downloading over the Internet. Data can be entered by any entity with access, including the patient, any care giver or other person with access. The data may be entered using the internet, a facsimile machine, or any other means of transporting information not mentioned. The Radiology departments most closely associated with the patient's management would likely upload data onto the website for others to download. A designated person or persons may evaluate the images based on objective, predetermined criteria.

Using interactive software, the images can be altered, and certain areas of the image(s) can be selected and manipulated into a more desirable (from a functional standpoint) configuration. Revision of the images may be outsourced, done by an automated computer program, constructed manually by qualified parties, or may be done through any other appropriate method.

The virtual-reworked image can be made available to authorized viewers, (and possibly by general viewers without identifiers). Coordinated with numerical data, caregivers and patients may use these images to make decisions about therapeutic options. The system may interface with existing, available programs used in assessing the organ or body part/system in terms of its functional status, including (but not limited to) viability, motion, density, cell metabolism, compliance, cellular function (e.g., oxygen exchange), relation to other structures, uptake of therapeutic or diagnostic substances, or other indicators that may be useful in diagnosis, treatment, or prognostic considerations.

More sophisticated usage of the reworked images may allow virtual sizing and shaping of devices used in a surgical procedure. This aspect can allow fabrication of a customized device for each individual patient, merely by analyzing the images downloaded and virtually remodeled. For example, this system may allow custom sizing of a shaping device 10 for left ventricular reconstruction and a patch to reconstruct the ventricular wall, both based on virtual three dimensional modeling for that individual patient.

A consultation team can evaluate, compare, and be available to help the patient and his/her caregivers make optimal use of the information. This team may function to assess the efficacy of treatment alternatives, once adequate data points are entered.

While this system may initially be applied to a cardiac platform, it is anticipated that broad applications in healthcare will follow. For example, the system could be useful for bone or joint reconstruction, identification of functional status of specific regions of emphysematous lungs, operations for morbid obesity, or noninvasive, virtual analysis of the stomach, as well as many other applications. It may also be useful as a teaching tool or training mechanism for instructors and students remote from each other.

Broad potential applications may also be developed in non-medical endeavors, where the pre-intervention status of any physical entity may be assessed and virtually manipulated at a central storage site with access to remote qualified users.

Although the foregoing invention has been described in terms of certain embodiments, other embodiments will be apparent to those of ordinary skill in the art from the disclosure herein. Additionally, other combinations, omissions, substitutions and modifications will be apparent to the skilled artisan in view of the disclosure herein. Accordingly, the present invention is not intended to be limited by the description of the preferred embodiments but is to be defined by reference to the appended claims.

Additionally, all patents and patent applications mentioned in this specification are herein incorporated by reference to the same extent as if each individual publication, patent, or patent application was specifically and individually indicated to be incorporated by reference.

1. A method of matching a shaper and a corresponding patch in a kit for treating ischemic congestive heart failure comprising the steps of:
selecting a shaper of an appropriate size for a patient; and
selecting a patch of an appropriate size for said patient, wherein said patch size is determined at least in part according to the shaper selected.

2. A kit for treating ischemic congestive heart failure comprising a shaping device and a patch, wherein said shaping device is appropriately sized for a patient and wherein said patch is sized according to the size of the shaping device such that said patch is also appropriately sized for said patient.

3. The kit of claim 2 further comprising suture having a plurality of sections wherein at least one of said sections comprises a superelastic or shape memory material.

4. A kit for treating ischemic congestive heart failure comprising a shaping device and a suture, wherein said shaping device is appropriately sized for a patient and wherein said suture is sized according to the size of the shaping device such that said suture is also appropriately sized for said patient.

5. A kit for treating ischemic congestive heart failure comprising a patch and a suture, wherein said patch is appropriately sized for a patient and wherein said suture is sized according to the size of the patch such that said suture is also appropriately sized for said patient.

6. A method for treating ischemic congestive heart failure in a patient comprising the steps of:
identifying akinetic tissue within a heart chamber wall;
making an incision through the akinetic tissue in the chamber wall;
inserting a shaping device and a patch into the chamber through the incision, wherein said shaping device and patch are attached;
removing the shaping device; and
closing the incision.

7. The method of claim 6 further comprising the step of at least partially securing said patch to the chamber wall.

8. The method of claim 7, wherein said patch comprises a device to aid in securing the patch to the heart chamber wall.

9. The method of claim 8, wherein said device to aid in securing the patch comprises one or more barbs.

10. The method of claim 6, wherein said patch comprises a superelastic or shape memory material.

11. The method of claim 6, wherein said shaping device comprises a superelastic or shape memory material.

12. A method for treating ischemic congestive heart failure in a patient comprising the steps of:
inserting a deployment device into the patient's atrium;
guiding the deployment device through the patient's mitral valve into a patient's left ventricle;
inserting a shaping device through said deployment device into the left ventricle;
reshaping said left ventricle by bringing one or more walls of said left ventricle against said shaping device; and
removing said shaping device.

13. The method of claim 12, wherein said deployment device comprises a catheter.

14. An endoscopic method for treating ischemic congestive heart failure in a patient comprising the steps of:
identifying akinetic tissue within a heart chamber wall;
making an incision through the akinetic tissue in the chamber wall;
inserting a shaping device into the chamber through the incision;
positioning the shaping device against a first side of said heart chamber wall;
applying a clamp to a second side of said heart chamber wall such that said clamp draws said heart chamber wall closer to said shaping device;
removing said shaping device; and
closing the incision.

15. The method of claim 14, wherein said clamp comprises a superelastic or shape memory material.

16. The method of claim 14 further comprising the step of excluding the akinetic tissue.

17. The method of claim 14 further comprising the step of initiating a closing suture while the shaper is inside the chamber thereby bringing the kinetic tissue closer together to improve the size and shape of the chamber.

18. The method of claim 14 further comprising the step of at least partially securing to the chamber wall, a patch comprising a superelastic or shape memory material.

19. The method of claim 14, wherein the step of making an incision comprises using an endoscope with an incising tip.

20. The method of claim 14, wherein the step of inserting a shaping device comprises using an endoscope to place the shaping device within the chamber.

21. The method of claim 14, wherein the step of inserting a shaping device comprises moving the shaping device relative to a sheath so as to permit the shaping device to assume a natural size and shape.

22. The method of claim 21, wherein the shaping device can assume one of two or more sizes selectable by moving the shaping device different distances relative to the sheath.

23. The method of claim 14, wherein said clamp can remain in place on said heart chamber wall after the procedure has been completed.
